(19) United States
(12) Patent Application Publication
(10) Pub. No.: US 2023/0384297 A1
(43) Pub. Date: Nov. 30, 2023

(54) ELECTROTHERMAL FLOW-ENHANCED ELECTROCHEMICAL MAGNETO-IMMUNOSENSOR

(71) Applicant: William Marsh Rice University, Houston, TX (US)

(72) Inventors: Peter B. LILLEHOJ, Houston, TX (US); Jiran LI, Houston, TX (US)

(73) Assignee: William Marsh Rice University, Houston, TX (US)

(21) Appl. No.: 18/173,246

(22) Filed: Feb. 23, 2023

Related U.S. Application Data
(60) Provisional application No. 63/313,697, filed on Feb. 24, 2022.

(51) Int. Cl. G01N 33/543; G01N 33/58; B01L 3/00

(52) U.S. Cl. CPC G01N 33/54326; G01N 33/58; B01L 3/502761; B01L 2400/043

(57) ABSTRACT

A simple immunosensor for rapid and high sensitivity measurements of protein biomarkers, such as SARS-CoV-2 nucleocapsid protein, in serum. The assay is based on a unique sensing scheme utilizing dually labeled magnetic nanobeads for immunomagnetic enrichment and signal amplification. This immunosensor can be integrated onto a microfluidic chip, which offers the advantages of minimal sample and reagent consumption, simplified sample handling, and enhanced detection sensitivity.

An ultrafast magneto-immunosensor, which employs AC electrothermally driven flow (ACEF) for accelerated mass transport and enhanced immunocomplex formation, is developed for high sensitivity protein measurement in whole blood samples.

### CROSS-REFERENCE TO RELATED APPLICATIONS

This application claims priority to U.S. Provisional Patent Application Ser. No. 63/313,697 filed Feb. 24, 2022, the entire contents of which are incorporated by reference herein.

### STATEMENT REGARDING FEDERALLY SPONSORED RESEARCH OR DEVELOPMENT

This invention was made with government support under Grant No. R01AI113257 awarded by the National Institutes of Health and Grant No. 1350560 awarded by the National Science Foundation. The government has certain rights in the invention.

### BACKGROUND

### 1. Field

The disclosure relates generally to the field of molecular biology. More particularly, it concerns protein detection method

### 2. Related Art

Diagnostic tests based on the detection and quantification of protein biomarkers are used for several important clinical applications, such as medical screening, [1,2] disease diagnosis [3-5] and monitoring response to treatment [6-8]. Currently, the most common laboratory technique for sensitive, quantitative detection of protein biomarkers in biological fluids is enzyme-linked immunosorbent assay (ELISA), which is considered the clinical gold standard [9]. However, ELISA requires bulky equipment for sample purification (i.e., centrifugation) and involves multiple washing steps and lengthy incubation (˜1.5-3 h in total), making it labor-intensive, time-consuming and limited to laboratory settings [10,11]. Several groups have recently developed immunosensors for rapid quantification of SARS-CoV-2 antigens in biofluids. Fabiani et al. [65] demonstrated the detection of SARS-CoV-2 S1 and N proteins at concentrations as low as 19 ng/mL and 8 ng/mL, respectively, in saliva using an electrochemical immunosensor. Tan et al. [66] developed a microfluidic chemiluminescent ELISA platform that could detect SARSCoV-2 S1 and N proteins in 10× diluted serum in 40 min. Torrente Rodriguez et al. [67] reported a multiplexed electrochemical immunoassay capable of detecting SARS-CoV-2 N protein and SARS-CoV-2 S1 IgG and IgM in 100× diluted serum samples. While these immunosensors were successful in measuring SARS-CoV-2 antigens in biofluids samples, they could not achieve high sensitivity (pg/mL) or required high sample dilution.

Prior efforts have been carried out to achieve high sensitivity detection of protein biomarkers in whole blood without the need for sample purification. Joh et al. developed an inkjet-printed fluorescence immunoassay that could detect IL-6 in chicken blood with a lower limit of detection (LOD) of 10.9 pg/mL [12]. Zupančič et al. reported an electrochemical immunoassay for detecting sepsis biomarkers which exhibited a lower LOD of 24.7 pg/mL in 50% whole blood [13]. Minopoli et al. demonstrated the detection of Plasmodium falciparum lactate dehydrogenase (PfLDH) in diluted (1:100) whole blood using fluorescence immunosensor with a lower LOD of 0.6 pg/mL.[14] While these techniques are capable of detecting proteins in whole blood with high sensitivity, they involve multiple washing steps and lengthy (50 min-4 h) incubation, hindering their use for applications requiring fast turnaround times, such as on-site testing or point-of-care testing. The ability to achieve rapid protein detection with high analytical sensitivity in whole blood is hampered by inefficient mass transport and slow protein binding kinetics in the complex liquid matrix.[15] Various techniques have been demonstrated to enhance mass transport and kinetics in surface binding assays, such as the use of microfluidic flows to confine the sample to the sensor surface[16] or continuously refresh the sensor with fresh analyte.[17] While these methods are capable of increasing the analytical sensitivity and reducing the assay time, they require complicated fluidic systems or result in increased sample/reagent consumption. Alternatively, direct current (DC) electrokinetics[18] or alternating current (AC) electrokinetics[19-21] has been shown to be an effective technique for manipulating and separating biomolecules in small volume samples. However, electrokinetics typically requires high operating voltages, which can cause electrolysis, and its performance is highly dependent on the fluid properties (e.g., conductivity, viscosity).[22] For these reasons, electrokinetic-based fluid manipulation is less effective for complex biological matrices, such as whole blood or minimally diluted blood.

Alternating current electrothermal flow (ACEF) is an alternative technique for generating microflows in small volume samples where an AC electrical field is applied to planar electrodes, resulting in non-uniform Joule heating. This localized Joule heating gives rise to gradients in permittivity and conductivity of the fluid, which generates thermally driven fluid forces that leads to swirling flows.[23] In contrast to electrokinetic-driven flow, ACEF is compatible with a broader range of biological fluids and can offer greater control over fluid motion. Computational and experimental studies by Lu et al. revealed the essential role of buoyancy force in long-range ACEF motion in microchannels.[24] Numerical studies by Sigurdson et al. further showed that electrothermally induced micro-stirring inside microchannels can improve antigen-antibody binding for flow-through assays.[25] ACEF has also been shown to enhance the performance of electrical biosensors for the detection of nucleic acids[26] and proteins[27]; however, these approaches involve multiple incubation steps requiring more than 30 minutes and are unable to achieve single pg/mL sensitivity in whole blood. Thus, there is an unmet need for improved methods for the detection of proteins with a high sensitivity in a short period of time.

### SUMMARY

In certain embodiments, the present disclosure provides systems and methods for detecting target proteins, including pathogens. Particular embodiments include an electrothermal flow-enhanced electrochemical magneto-immunosensor. One embodiment of the present disclosure is a simple immunosensor for rapid and high sensitivity measurements of protein biomarkers, such as SARS-CoV-2 nucleocapsid protein, in serum.

In another embodiment, there is provided a microfluidic method for detecting a target protein in a sample comprising (a) contacting the sample with immunosensors comprising dually-labeled magnetic beads (DMBs) conjugated to a capture antibody specific for the target protein and an enzyme reporter; (b) loading the sample and DMBs into a microfluidic chip; (c) applying AC electrothermal flow (ACEF) to the sample to mix the sample; (d) performing immunomagnetic enrichment to generate an electrochemical signal; and (e) detecting the target protein by measuring levels of the reporter.

In some aspects, the capture antibody is a human monoclonal capture antibody. In certain aspects, the sample to DMBs ratio is about 10:1 to about 20:1, such as about 10:1, 11:1, 12:1, 13:1, 14:1, 15:1, 16:1, 17:1, 18:1, 19:1, or 20:1. In certain aspects, contacting is for about 40 minutes to about 60 minutes, such as about 50 minutes.

In some aspects, the sample is diluted serum. In certain aspects, contacting is performed for about 20 minutes to 30 minutes. In some aspects, the sample and DMBs are loaded onto the microfluidic chip using a capillary tube and plunger or a syringe pump. In some aspects, the microfluidic chip comprises a 400 um-high reaction chamber. In some aspects, the chamber encompasses the immunosensor to the inlet and outlet.

In certain aspects, the reporter generates an electrochemical signal. In some aspects, the reporter generates an optical signal. In particular aspects, the reporter is a chemiluminescent reporter. In some aspects, the reporter is horseradish peroxidase (HRP). In specific aspects, measuring levels of the reporter comprises using an HRP-conjugated detection antibody and detecting colorimetric signal. In certain aspects, the HRP-conjugated detection antibody is an HRP-conjugated rabbit monoclonal detection antibody. In some aspects, performing immunomagnetic enrichment comprises placing the microfluidic chip on a magnet. In some aspects, the microfluidic chip is placed on a magnet for about 30 seconds to 2 minutes. In specific aspects, the microfluidic chip is placed on a magnet for about 1 minute. In certain aspects, measuring levels of the reporter comprise detecting amperometric current. In some aspects, the method has a 50 pg/mL sensitivity, such as a 40 pg/mL, 30 pg/mL, 20 pg/mL, 10 pg/mL, 5 pg/mL, or 1 pg/mL.

In some aspects, the ACEF is applied at about 200 kHz and 25 Vpp. In certain aspects, the ACEF is applied for about 5 minutes. In some aspects, the target protein is a protein antigen, such as but not limited to SARS-CoV antigen or plasmodium falciparum histidine-rich proteins 2 (PfHRP2).

In certain aspects the sample is a biological fluid sample. In some aspects, the sample is a saliva, urine, or plasma sample. In some aspects, the sample is a serum sample. In certain aspects, the sample is a whole blood sample. In some aspects, the method does not comprise centrifugation of the sample. In particular aspects, the sample is an undiluted sample. In other aspects, the whole blood sample is diluted by a 5× dilution factor. the method has a 5 pg/mL sensitivity.

In some aspects, the method is performed in less than 1 hour, such as less than 50 minutes, less than 40 minutes, less than 30 minutes, less than 20 minutes, or less than 10 minutes. In particular aspects, the sample volume is less than 50 uL, such as less than 40 uL, less than 30 uL, or less than 20 uL.

A further embodiment provides a device for quantitative measurements of a target protein in a sample, wherein the device is a handheld diagnostic comprising a microfluidic chip with an immunosensor; and a magnet proximal to the immunosensor.

In some aspects, the microfluidic chip further comprises an inlet and a sample loading mechanism. In certain aspects, the microfluidic chip further comprises an outlet. In some aspects, the microfluidic chip further comprises a waste reservoir. In particular aspects, the immunosensor comprises a working electrode, a counter electrode and a reference electrode. In some aspects, the device is configured to provide mixing to a sample via alternating current electrothermal flow (ACEF).

In further aspects, the device further comprises a detector configured to detect a signal from the immunosensor. In some aspects, the detector is an electrochemical analyzer configured to detect an amperometric current signal. In particular aspects, the detector is an optical detector configured to detect a colorimetric signal. In additional aspects, the device further comprises a smart phone and multi-channel potentiostat.

In yet another embodiment, there is provided a method for treating a coronavirus infection comprising administering an effective amount of an antiviral to a subject identified to have a coronavirus infection by the method of the present embodiments or aspects thereof (e.g, a microfluidic method for detecting a target protein in a sample comprising (a) contacting the sample with immunosensors comprising dually-labeled magnetic beads (DMBs) conjugated to a capture antibody specific for the target protein and an enzyme reporter; (b) loading the sample and DMB s into a microfluidic chip; (c) applying AC electrothermal flow (ACEF) to the sample to mix the sample; (d) performing immunomagnetic enrichment to generate an electrochemical signal; and (e) detecting the target protein by measuring levels of the reporter). In some aspects, the antiviral is paxlovid, molnupiravir, or remdesivir.

Another embodiment provides a microfluidic electrochemical magneto-immunosensor for rapid and high sensitivity measurements of protein biomarkers in biofluid samples, wherein the assay is based on a sensing scheme utilizing dually labeled magnetic nanobeads for immunomagnetic enrichment and signal amplification.

A further embodiment provides a microfluidic electrochemical magneto-immunosensor according to the present embodiments and aspects thereof integrated onto a microfluidic chip.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature and not as restrictive. Additional objects and advantages of the invention will be set forth in part in the description which follows and in part will be obvious from the description or may be learned by practice of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. For a more complete understanding of the present invention and the advantages thereof, reference is now made to the following description and the accompanying drawings, in which:

FIG. 1A is a schematic illustration of a microfluidic immunosensor chip highlighting the magnetic concentration of DMBs to the sensor surface.

FIG. 1B is a schematic illustration of microfluidic immunosensor chip for the smartphone-based diagnostic device.

FIG. 1C is a schematic illustration of experimental setup and electrochemical sensing scheme using the PalmSens4-based sensing platform.

FIG. 2A is a graph of amperometric currents generated from undiluted serum samples spiked with SARS-CoV-2 N protein at 0 and 1 ng/mL and corresponding S/B ratios using immunosensors with five different SARS-CoV-2 N protein antibody pairs. Measurements were performed using magnetic enrichment and incubation times of 1 and 50 min, respectively.

FIG. 2B is a graph of amperometric currents generated from undiluted serum samples spiked with SARS-CoV-2 N protein at 0 and 1 ng/mL and corresponding S/B ratios with varying sample/DMB volume ratios. Measurements were performed using magnetic enrichment and incubation times of 1 and 50 min, respectively.

FIG. 2C is a graph of amperometric currents generated from undiluted serum samples spiked with the SARS-CoV-2 N protein at 0 and 1 ng/mL and corresponding S/B ratios with varying magnetic enrichment times and a 50 min sample incubation duration.

FIG. 2D is a graph of amperometric currents generated from undiluted serum samples spiked with SARS-CoV-2 N protein at 0 and 1 ng/mL and corresponding S/B ratios with varying incubation times and 1 min of magnetic enrichment. Each bar represents the mean±standard deviation (SD) of three separate measurements obtained using new sensors.

FIG. 3A is a graph of chronoamperograms generated from whole serum samples spiked with SARS-CoV-2 N protein at varying concentrations.

FIG. 3B illustrates calibration plots based on amperometric currents at 100 s for whole serum samples with 50 min incubation and 5× diluted serum samples with 25 min incubation. Each data point represents the mean±SD of three separate measurements obtained using new sensors. The inset shows amperometric currents for samples containing SARS-CoV-2 N protein from 0 to 1 ng/mL. Each bar represents the mean±SD of three separate measurements obtained using new sensors. The dashed and solid lines correspond to the lower LOD for measurements of whole serum and 5× diluted serum, respectively.

FIG. 3C is a graph of amperometric currents generated from serum samples containing SARS-CoV-2 N protein, SARS-CoV N protein, MERS-CoV N protein, SARS-CoV-2 Spike RBD protein and nonspiked serum (blank control). Each bar represents the mean±SD of three separate measurements obtained using new sensors.

FIG. 4A shows a smartphone-based diagnostic device for electrochemical measurements of SARS-CoV-2 N protein in accordance with an embodiment of the present disclosure.

FIG. 4B shows a microfluidic immunosensor chip consisting of a cAb-coated SPGE sensor and PET-PMMA cartridge in accordance with an embodiment of the present disclosure.

FIG. 4C is a graph of calibration plots based on amperometric currents at 100 s for whole serum samples with 50 min incubation and 5× diluted serum samples with 25 min incubation. Each data point represents the mean±SD of three separate measurements obtained using new sensors. The inset shows amperometric currents for samples containing SARS CoV-2 N protein from 0 to 1 ng/mL. Each bar represents the mean±SD of three separate measurements obtained using new sensors. The dashed and solid lines correspond to the lower LOD for measurements of whole serum and 5× diluted serum, respectively. MERS-CoV N protein, SARS-CoV-2 Spike RBD protein and nonspiked serum (blank control). Each bar represents the mean±SD of three separate measurements obtained using new sensors.

FIG. 5A is a graph of electrochemical signals generated from serum specimens obtained from COVID-19 patients (positive) and uninfected individuals (negative). Each bar represents the mean±SD of three separate measurements obtained using new sensors.

FIG. 5B is a graph of calculated SARS-CoV-2 N protein concentration and corresponding S/B ratios for clinical serum specimens.

FIG. 6 is a graph of amperometric currents generated from 5× diluted serum samples (25 μL) spiked with SARS-CoV-2 N protein at 0 ng/mL, 1 ng/mL and 10 ng/mL and corresponding S/B ratios using a microfluidic immunosensor and an open well immunosensor. Each bar represents the mean±SD of three separate measurements obtained using new sensors.

FIG. 7 illustrates a design and working principle of the ACEF-enhanced magneto-immunosensor. (A) Schematic illustration of the blood sample premixed with dually-labeled nanobeads (DMBs) on the screen-printed gold electrode (SPGE) sensor. Upon application of an AC potential between the working electrode (WE) and counter electrode (CE), swirling microflows are generated within the droplet due to electrothermally induced forces, enhancing the transport of proteins and DMBs in the sample and promoting the formation of antigen-DMB immunocomplexes. (B) Schematic depicting the magnetic concentration (MC) of antigen-DMB immunocomplexes on the capture antibody-immobilized sensor surface, which is achieved by placing the SPGE sensor on a permanent magnet. (C) Schematic illustration of the electrochemical (EC) sensing scheme after the SPGE sensor has been rinsed and loaded with TMB substrate. Horseradish peroxidase immobilized on the DMBs catalyzes the reduction of H2O2 coupled to TMB oxidation. The oxidized TMB is reduced upon the application of a bias potential between the WE and CE, which generates an amperometric current that is proportional to the concentration of target antigen attached to the sensor surface.

FIG. 8. Illustrates the influence of blood dilution on the sensor performance. (A) Amperometric currents generated from whole blood spiked with PfHRP2 at 0 ng/mL and 1 ng/mL and corresponding ΔI values with different sample dilution factors (0×, 2×, 5×, and 20×). Each bar represents the mean±SD of five replicate measurements using new sensors. (B) ΔI values generated from whole blood spiked with PfHRP2 at 0 ng/mL and 1 ng/mL obtained from five independent blood donor and with different sample dilution factors (0×, 2×, 5×, and 20×).

FIG. 9 illustrates characterization of ACEF mixing. (A) Optical images of 60 μL, 80 μL and 100 μL blood droplets on the SPGE sensor and corresponding 2D COMSOL simulation results of the velocity profile with ACEF mixing (25 Vpp, 200 kHz, 5 min). (B) Amperometric currents generated from 5× diluted whole blood spiked with PfHRP2 at 0 ng/mL and 1 ng/mL and corresponding ΔI values with different sample volumes. (C) Sequential still frame images showing the motion of 6 μm red polystyrene beads in an 80 μL 1% BSA in 1×PBS droplet with and without ACEF mixing. (D) Amperometric currents generated from 5× diluted whole blood spiked with PfHRP2 at 0 ng/mL and 1 ng/mL and corresponding ΔI values for different ACEF potentials and durations. Each bar represents the mean±SD of three replicate measurements obtained using new sensors. (E) Experimental setup for performing ACEF mixing and thermal image of an 80 μL blood sample on the SPGE sensor after 5 min of ACEF mixing (25 Vpp, 200 kHz).

FIG. 10 illustrates performance of the ACEF-enhanced magneto-immunosensor for quantifying PfHRP2 in spiked and clinical blood samples. (A) Amperometric currents generated from 5× diluted whole blood spiked with PfHRP2 at 0 ng/mL and 1 ng/mL and corresponding ΔI values using different sensor enhancement methods. Each bar represents the mean±SD of three replicate measurements obtained using new sensors. (B) Chronoamperograms generated from 5× diluted whole blood spiked with PfHRP2 at concentrations from 0 to 5,000 pg/mL with ACEF mixing and magnetic concentration. (C) Calibration plot based on amperometric currents at 60 s obtained from chronoamperograms in panel B. Inset shows amperometric currents for samples containing PfHRP2 from 0 to 100 pg/mL. Each bar represents the mean±SD of three replicate measurements obtained using new sensors. The dashed line corresponds to the lower limit of detection, calculated as 3× the SD of the amperometric current at zero concentration divided by the slope of the calibration curve. (D) PfHRP2 levels in clinical blood samples measured by the ACEF-enhanced magneto-immunosensor and a commercial PfHRP2 ELISA kit. (E) Amperometric signals generated by the ACEF-enhanced magneto-immunosensor and absorbance values (OD 450 nm) generated by ELISA for paired blood samples obtained from individuals with P. falciparum infection (n=8) and healthy, uninfected individuals (n=6)

FIG. 11 illustrates optimization of the magneto-immunosensor. (A) Amperometric currents generated from 5× diluted whole blood spiked with PfHRP2 at 0 ng/mL and 1 ng/mL and corresponding ΔI values with varying sample to DMB volume ratios, a 15 min pre-magnetic concentration (MC) incubation duration and 1 min of MC. (B) Amperometric currents generated from 5× diluted whole blood spiked with PfHRP2 at 0 ng/mL and 1 ng/mL and corresponding ΔI values with varying pre-MC incubation durations and 1 min of MC. (C) Amperometric currents generated from 5× diluted whole blood spiked with PfHRP2 at 0 ng/mL and 1 ng/mL and corresponding ΔI values with different incubation conditions, a 15 min pre-MC incubation duration and 1 min of MC. (D) Amperometric currents generated from 5× diluted whole blood spiked with PfHRP2 at 0 ng/mL and 1 ng/mL and corresponding ΔI values with varying MC durations and a 15 min pre-MC incubation duration. Each bar represents the mean±SD of three replicate measurements obtained using new sensors.

FIG. 12 illustrates immunosensor performance using varying sample dilution factors. (A) Amperometric currents generated from undiluted whole blood spiked with PfHRP2 at 0 ng/mL and 1 ng/mL and corresponding ΔI values from five independent blood donors. (B) Amperometric currents generated from 2× diluted whole blood with PfHRP2 at 0 ng/mL and 1 ng/mL and corresponding ΔI values from five independent blood donors. (C) Amperometric currents generated from 5× diluted whole blood spiked with PfHRP2 at 0 ng/mL and 1 ng/mL and corresponding ΔI values from five independent blood donors. (D) Amperometric currents generated from 20× diluted whole blood spiked with PfHRP2 at 0 ng/mL and 1 ng/mL and corresponding ΔI values from five independent blood donors. Each bar represents the mean±SD of three replicate measurements obtained using new sensors.

FIG. 13 illustrates optimization of assay parameters for the ACEF-enhanced magneto-immunosensor. (A) Amperometric currents generated from 5× diluted whole blood spiked with PfHRP2 at 0 ng/mL and 1 ng/mL and corresponding ΔI values with ACEF mixing (20 Vpp) at varying mixing durations. (B) Amperometric currents generated from 5× diluted whole blood spiked with PfHRP2 at 0 ng/mL and 1 ng/mL and corresponding ΔI values with ACEF mixing (25 Vpp) at varying mixing durations. (C) Amperometric currents generated from 5× diluted whole blood spiked with PfHRP2 at 0 ng/mL and 1 ng/mL and corresponding ΔI values with ACEF mixing (30 Vpp) at varying mixing durations. Each bar represents the mean±standard deviation (SD) of three replicate measurements obtained using new sensors.

FIG. 14 illustrates droplet temperature after ACEF mixing. Thermal images of an 80 μL blood sample on the SPGE sensor after 5 min of ACEF mixing at 20 Vpp, 25 Vpp or 30 Vpp and 200 kHz.

FIG. 15 illustrates selectivity of the immunosensor. Amperometric currents generated from 5× diluted whole blood containing 1 ng/mL of PfHRP2, PfLDH or pan-Plasmodium aldolase (aldolase), and non-spiked blood (negative control). Each bar represents the mean±SD of three replicate measurements obtained using new sensors.

FIG. 16 illustrates a schematic of the magneto-ELISA testing protocol. (A) Incubation of the sample with DMPs. (B) Magnetic concentration of antigen-DMP immunocomplexes on cAb-immobilized wells. (C) Generation of the colorimetric signal due to the HRP-catalyzed oxidation of TMB.

FIG. 17 illustrates enhancement of ELISA with DMPs and magnetic concentration. (A) Signal-to-background ratios generated from PfHRP2-spiked human serum samples using magnetic particles labeled with HRP-conjugated dAb and free HRP (beige) or beads labeled with HRP-conjugated dAb only (blue). Each bar represents the mean of three measurements. (B) Signal-to-background ratios generated from PfHRP2-spiked human serum samples with 1 min of magnetic concentration (beige) or with 30 min incubation without magnetic concentration (blue). Each bar represents the mean of four measurements.

FIG. 18 illustrates optimization of assay parameters. (A) Absorbance values generated from human serum spiked with 1 ng mL⁻¹ or 0 ng mL⁻¹ of PfHRP2 using different antibody pairs. (B) Absorbance values generated from PfHRP2-spiked human serum with varying magnetic concentration (MC) durations, 14 min of sample-DMP incubation, and 5 min of post-MC incubation. (C) Absorbance values generated from PfHRP2-spiked human serum with varying sample-DMP incubation durations, 1 min of MC, and 5 min of post-MC incubation. (D) Absorbance values generated from PfHRP2-spiked human serum with varying durations of post-MC incubation, 14 min of sample-DMP incubation, and 1 min of MC. Each bar represents the mean±SD of four measurements. * indicates p<0.05, ** indicates p<0.01.

FIG. 19 illustrates analytical performance of the magneto-ELISA. (A) Calibration curve generated from absorbance values measured at varying PfHRP2 concentrations from 0-1 ng mL⁻¹ in human serum. Inset shows absorbance values at 0 and 0.01 ng mL⁻¹ PfHRP2. (B) Absorbance values generated from human serum spiked with 1 ng mL⁻¹ of PfHRP2, Plasmodium aldolase, PfLDH, or nonspiked sera. (C) Absorbance values at varying PfHRP2 concentrations from 0-1 ng mL⁻¹ in diluted serum, plasma, or blood samples. (D) Calibration curve generated from absorbance values measured at varying SARS-CoV-2 N protein concentrations from 0-1 ng mL⁻¹ in human serum. Inset shows absorbance values at 0 and 0.01 ng mL⁻¹ SARS-CoV-2 N protein. Each data point and bar represents the mean±SD of at least three measurements.

FIG. 20 illustrates validation of the magneto-ELISA. Concentration of PfHRP2 in malaria positive (n=13) and RDT negative (n=6) whole blood samples determined using the magneto-ELISA and a commercial PfHRP2 ELISA kit. Each point represents the mean of two (malaria-positive) or three (malaria-negative) measurements. The inset shows individual samples with PfHRP2 concentrations between 0-5 ng mL⁻¹.

FIG. 21 illustrates a design of the magnetic stage. (A) Dimensions of the PMMA base for the magnet array. All units are in mm. (B) Photograph of assembled magnet stage.

FIG. 22 illustrates optimization of magneto-ELISA parameters. (A) Absorbance values generated from PfHRP2-spiked human sera using different sample-to-DMP volume ratios. (B) Absorbance values generated from PfHRP2-spiked human sera (1 ng/mL) using varying sized magnetic particles. (C) Absorbance values generated from human sera spiked with 1 ng/mL or 0 ng/mL of PfHRP2 using static or agitated incubation, at varying speeds, at room temperature before magnetic concentration. All measurements were performed with 14 min of sample-DMP incubation, 1 min of magnet concentration, and 5 min of post-magnetic concentration incubation. Each bar represents the mean±SD of four measurements. * indicates p<0.05, ** indicates p<0.01.

### DETAILED DESCRIPTION OF THE PREFERRED EMBODIMENTS

Magnetic beads are widely used in immunoassays for biomolecular separation and enrichment.[28,29] Prior reports have demonstrated electrochemical sensors employing magnetic beads for rapid, quantitative biomolecular detection.[30-32] However, these platforms require multiple sample processing steps and were limited to purified serum samples. In previous work, it was shown that the use of magnetic nanobeads combined with immunomagnetic enrichment could generate an amplified electrochemical signal, enabling high sensitivity electrochemical detection.[33] However, like many immunosensors, this approach still involved lengthy (≥1 hr) incubation and required purified serum samples for high sensitivity measurements. To address these limitations, a rapid, highly sensitive magneto-immunosensor was developed that employs ACEF mixing for accelerated mass transport and immunocomplex formation. This immunosensor utilizes dually-labeled magnetic nanobeads (DMBs) that are coated with a detection antibody and enzyme reporter to form immunocomplexes with the target protein, allowing for simplified immunomagnetic enrichment and increased signal amplification. The present studies showed that ACEF mixing enhances biomolecular transport and promotes immunocomplex formation, enabling high sensitivity detection at single pg/mL (<100 fM) levels without requiring sample purification or lengthy incubation. Proof of concept was demonstrated by detecting Plasmodium falciparum histidine-rich protein 2 (PfHRP2), a biomarker for P. falciparum, which accounts for >90% of global fatalities due to malaria infection.[34] Measurements of PfHRP2 in clinical blood samples obtained from malaria-infected individuals revealed that this immunosensor offers greater diagnostic accuracy than a commercial PfHRP2 ELISA kit, while being much faster and simpler to perform.

In the present studies, it was demonstrated for the first time rapid (<1 h), high sensitivity measurements of SARS-CoV-2 N protein in whole (undiluted) serum. This unique immunosensor utilizes dually-labeled magnetic nanobeads (DMBs) for on-chip immunomagnetic enrichment and signal amplification. Several assay parameters, including the antibody pair, the volume ratio of the sample to magnetic beads, the magnetic enrichment time, and the incubation time, were optimized to enhance the detection sensitivity. The capability of this immunoassay to detect SARS-CoV-2 N protein in undiluted human serum samples in <1 h was shown to have pg/mL sensitivity. It was also demonstrated that the SARS-CoV-2 N protein can be detected in serum samples using a smartphone-based diagnostic device that can achieve high sensitivity and reproducibility. Lastly, the utility of this platform was demonstrated for accurately detecting COVID-19 infection by performing measurements of clinical serum specimens from COVID-19 patients and healthy, uninfected individuals.

In some aspects, the presented assay is based on a unique sensing scheme utilizing dually labeled magnetic nanobeads for immunomagnetic enrichment and signal amplification. This immunosensor is integrated onto a microfluidic chip, which offers the advantages of minimal sample and reagent consumption, simplified sample handling, and enhanced detection sensitivity. The functionality of this immunosensor was validated by using it to detect SARS-CoV-2 nucleocapsid protein, which could be detected at concentrations as low as 50 pg/mL in whole serum and 10 pg/mL in 5× diluted serum. The present assay may be performed with a handheld smartphone-based diagnostic device that could detect SARS-CoV-2 nucleocapsid protein at concentrations as low as 230 pg/mL in whole serum and 100 pg/mL in 5× diluted serum. Lastly, the capability of this immunosensor was assessed to diagnose COVID-19 infection by testing clinical serum specimens, which revealed its ability to accurately distinguish PCR-positive COVID-19 patients from healthy, uninfected individuals based on SARS-CoV 2 nucleocapsid protein serum levels. This work is the first demonstration of rapid (<1 h) SARS-CoV-2 antigen quantification in whole serum samples. The ability to rapidly detect SARS-CoV-2 protein biomarkers with high sensitivity in very small (<50 μL) serum samples makes this platform a promising tool for point-of-care COVID-19 testing.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “ and/or” unless explicitly indicated to refer to alternatives only or the alternatives are mutually exclusive, although the disclosure supports a definition that refers to only alternatives and “and/or.” As used herein “another” may mean at least a second or more.

The term “about” means, in general, within a standard deviation of the stated value as determined using a standard analytical technique for measuring the stated value. The terms can also be used by referring to plus or minus 5% of the stated value.

The phrase “effective amount” or “therapeutically effective” means a dosage of a drug or agent sufficient to produce a desired result. The desired result can be subjective or objective improvement in the recipient of the dosage, increased lung growth, increased lung repair, reduced tissue edema, increased DNA repair, decreased apoptosis, a decrease in tumor size, a decrease in the rate of growth of cancer cells, a decrease in metastasis, or any combination of the above.

As used herein, the term “antibody” refers to an immunoglobulin, derivatives thereof which maintain specific binding ability, and proteins having a binding domain which is homologous or largely homologous to an immunoglobulin binding domain These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. The antibody may be a bi-specific antibody. In exemplary embodiments, antibodies used with the methods and compositions described herein are derivatives of the IgG class. The term antibody also refers to antigen-binding antibody fragments. Examples of such antibody fragments include, but are not limited to, Fab, Fabÿ, F(abÿ)2, scFv, Fv, dsFv diabody, and Fd fragments. Antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 10 amino acids and more typically will comprise at least about 200 amino acids.

“Subject” and “patient” refer to either a human or non-human, such as primates, mammals, and vertebrates. In particular embodiments, the subject is a human

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a tumor or malignancy, delay or slowing of tumor growth and/or metastasis, and an increased lifespan as compared to that expected in the absence of treatment.

The term “determining an expression level” as used herein means the application of a gene specific reagent such as a probe, primer or antibody and/or a method to a sample, for example a sample of the subject and/or a control sample, for ascertaining or measuring quantitatively, semi-quantitatively or qualitatively the amount of a gene or genes, for example the amount of mRNA. For example, a level of a gene can be determined by a number of methods including for example immunoassays including for example immunohistochemistry, ELISA, Western blot, immunoprecipitation and the like, where a biomarker detection agent such as an antibody for example, a labeled antibody, specifically binds the biomarker and permits for example relative or absolute ascertaining of the amount of polypeptide biomarker, hybridization and PCR protocols where a probe or primer or primer set are used to ascertain the amount of nucleic acid biomarker, including for example probe based and amplification based methods including for example microarray analysis, RT-PCR such as quantitative RT-PCR, serial analysis of gene expression (SAGE), Northern Blot, digital molecular barcoding technology, for example Nanostring:nCounter™ Analysis, and TaqMan quantitative PCR assays. Other methods of mRNA detection and quantification can be applied, such as mRNA in situ hybridization in formalin-fixed, paraffin-embedded (FFPE) tissue samples or cells. This technology is currently offered by the QuantiGene®ViewRNA (Affymetrix), which uses probe sets for each mRNA that bind specifically to an amplification system to amplify the hybridization signals; these amplified signals can be visualized using a standard fluorescence microscope or imaging system. This system for example can detect and measure transcript levels in heterogeneous samples; for example, if a sample has normal and tumor cells present in the same tissue section. As mentioned, TaqMan probe-based gene expression analysis (PCR-based) can also be used for measuring gene expression levels in tissue samples, and for example for measuring mRNA levels in FFPE samples. In brief, TaqMan probe-based assays utilize a probe that hybridizes specifically to the mRNA target. This probe contains a quencher dye and a reporter dye (fluorescent molecule) attached to each end, and fluorescence is emitted only when specific hybridization to the mRNA target occurs. During the amplification step, the exonuclease activity of the polymerase enzyme causes the quencher and the reporter dyes to be detached from the probe, and fluorescence emission can occur. This fluorescence emission is recorded and signals are measured by a detection system; these signal intensities are used to calculate the abundance of a given transcript (gene expression) in a sample.

The term “sample” as used herein includes any biological specimen obtained from a patient. Samples include, without limitation, whole blood, plasma, serum, red blood cells, white blood cells (e.g., peripheral blood mononuclear cells), ductal lavage fluid, nipple aspirate, lymph (e.g., disseminated tumor cells of the lymph node), bone marrow aspirate, saliva, urine, stool (i.e., feces), sputum, bronchial lavage fluid, tears, fine needle aspirate (e.g., harvested by fine needle aspiration that is directed to a target, such as a tumor, or is random sampling of normal cells, such as periareolar), any other bodily fluid, a tissue (e.g., tumor tissue) such as a biopsy of a tumor (e.g., needle biopsy) or a lymph node (e.g., sentinel lymph node biopsy), and cellular extracts thereof. In some embodiments, the sample is whole blood or a fractional component thereof such as plasma, serum, or a cell pellet.

The terms “increased”, “elevated”, “overexpress”, “overexpression”, “overexpressed”, “up-regulate”, or “up-regulated” interchangeably refer to a biomarker that is present at a detectably greater level in a biological sample, e.g. plasma, from a patient with cancer, in comparison to a biological sample from a patient without cancer. The term includes overexpression in a sample from a patient with cancer due to transcription, post-transcriptional processing, translation, post-translational processing, cellular localization (e.g, organelle, cytoplasm, nucleus, cell surface), and RNA and protein stability, as compared to a sample from a patient without cancer. Overexpression can be detected using conventional techniques for detecting mRNA (i.e., RT-PCR, PCR, hybridization) or proteins (i.e., ELISA, immunohistochemical techniques, mass spectroscopy, Luminex® xMAP technology). Overexpression can be 10%, 20%, 30%, 40%, 50%, 60%, 70%, 80%, 90% or more in comparison to a sample from a patient without cancer. In certain instances, overexpression is 1-fold, 2-fold, 3-fold, 4-fold 5, 6, 7, 8, 9, 10, or 15-fold or more higher levels of transcription or translation in comparison to a sample from a patient without cancer.

A “label,” “imaging agent”” or a “detectable moiety” is a composition detectable by spectroscopic, photochemical, biochemical, immunochemical, chemical, or other physical means. For example, useful labels include 32 P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

As used herein, the term “biomarker” refers to any biological feature from tissue sample or a cell to be identified or quantitated. A biomarker can be useful or potentially useful for measuring the initiation, progression, severity, pathology, aggressiveness, grade, activity, disability, mortality, morbidity, disease sub-classification or other underlying feature of one or more biological processes, pathogenic processes, diseases, or responses to a therapeutic intervention. A biomarker is virtually any biological compound, such as a protein and a fragment thereof, a peptide, a polypeptide, a proteoglycan, a glycoprotein, a lipoprotein, a carbohydrate, a lipid, a nucleic acid, an organic on inorganic chemical, a natural polymer, and a small molecule, that is present in the sample to be analyzed and that can be isolated from, or measured in, the sample.

As used herein, the term “detecting” refers to observing a signal from a label moiety to indicate the presence of a biomarker in the sample. Any method known in the art for detecting a particular detectable moiety can be used for detection. Exemplary detection methods include, but are not limited to, spectroscopic, photochemical, biochemical, immunochemical, electrical, optical or chemical methods.

As used herein “ACEF mixing” refers to mixing of fluids via alternating current electrothermal flow (ACEF).

### II. METHODS OF DETECTION

### A. Biological Sample

Certain embodiments of the present disclosure concern the detection and quantification of the expression of certain antigens or biomarkers in a sample. As used herein, the term “biological sample” may refer to a whole organism or a subset of its tissues, cells or component parts. A “biological sample” may also refer to a homogenate, lysate, or extract prepared from a whole organism or a subset of its tissues, cells or component parts, or a fraction or portion thereof. Typically, the biological sample is diluted prior to performing an assay. Non-limiting examples of biological samples include urine, blood, cerebrospinal fluid (CSF), pleural fluid, sputum, and peritoneal fluid, bladder washings, secretions, oral washings, tissue samples, touch preps, or fine-needle aspirates. The sample may comprise body fluids and tissue samples that include but are not limited to blood, tissue biopsies, spinal fluid, meningeal fluid, urine, alveolar fluid. In some embodiments, a biological sample may be a cell line, cell culture or cell suspension. Preferably, a biological sample corresponds to the amount and type of DNA and/or expression products present in a parent cell from which the sample was derived. A biological sample can be from a human or non-human subject. In particular embodiments, the sample is a plasma sample, serum sample, or whole blood sample. The assay may also be applied to in vivo tissue, such as during a surgery.

### B. Detection Methods

The level of expression of the biomarker may be measured by the present rapid, highly sensitive magneto-immunosensor method employing ACEF mixing for accelerated mass transport and immunocomplex formation. The present immunosensor method utilizes dually-labeled magnetic nanobeads (DMBs) that are coated with a detection antibody and enzyme reporter to form immunocomplexes with the target protein, allowing for simplified immunomagnetic enrichment and increased signal amplification. The present studies showed that ACEF mixing enhances biomolecular transport and promotes immunocomplex formation, enabling high sensitivity detection at single pg/mL (<100 fM) levels without requiring sample purification or lengthy incubation.

Other methods of detection include ELISA, western blotting, mass spectrometry, a capillary immune-detection method, isoelectric focusing, an immune precipitation method or immunohistochemistry, antibody-based optical imaging, ultrasound imaging, MRI imaging, PET imaging, and phototherapy.

An enzyme-linked immunosorbent assay, or ELISA, may be used to measure the differential expression of a plurality of biomarkers. There are many variations of an ELISA assay. ELISA tests may be formatted for direct, indirect, competitive, or sandwich detection of the analyte. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. The primary antibodies are conjugated to a detection system, such as an enzyme that produces a detectable product. The antibody-antibody complexes may be detected indirectly. For example, the primary antibody is detected by a secondary antibody that is conjugated to a detection system, as described above. The microtiter plate is then scanned and the raw intensity data may be converted into expression values using means known in the art. Single- and Multi-probe kits are available from commercial suppliers, e.g., Meso Scale Discovery (MSD).

In one ELISA method, a first, or capture, binding agent, such as an antibody that specifically binds the biomarker of interest, is immobilized on a suitable solid phase substrate or carrier. The test biological sample is then contacted with the capture antibody and incubated for a desired period of time. After washing to remove unbound material, a second, detection, antibody that binds to a different, non-overlapping, epitope on the biomarker is then used to detect binding of the polypeptide biomarker to the capture antibody. The detection antibody is preferably conjugated, either directly or indirectly, to a detectable moiety. Examples of detectable moieties that can be employed in such methods include, but are not limited to, cheminescent and luminescent agents; fluorophores such as fluorescein, rhodamine and eosin; radioisotopes; colorimetric agents; and enzyme-substrate labels, such as biotin.

In another embodiment, the ELISA is a competitive binding assay, wherein labeled biomarker is used in place of the labeled detection antibody, and the labeled biomarker and any unlabeled biomarker present in the test sample compete for binding to the capture antibody. The amount of biomarker bound to the capture antibody can be determined based on the proportion of labeled biomarker detected.

In certain embodiments, the biomarker or antibody bound to the biomarker is directly or indirectly labeled with a detectable moiety. The role of a detectable agent is to facilitate the detection step of the diagnostic method by allowing visualization of the complex formed by binding of the binding agent to the protein marker (or fragment thereof). The detectable agent can be selected such that it generates a signal that can be measured and whose intensity is related (preferably proportional) to the amount of protein marker present in the sample being analyzed. Methods for labeling biological molecules such as polypeptides and antibodies are well-known in the art. Any of a wide variety of detectable agents can be used in the practice of the present disclosure. Suitable detectable agents include, but are not limited to: various ligands, radionuclides, fluorescent dyes, chemiluminescent agents, microparticles (such as, for example, quantum dots, nanocrystals, phosphors and the like), photosensitizers, enzymes (such as, those used in an ELISA, i.e., horseradish peroxidase, beta-galactosidase, luciferase, alkaline phosphatase), colorimetric labels, magnetic labels, and biotin, digoxigenin or other haptens and proteins for which antisera or monoclonal antibodies are available.

The antibodies may be attached to imaging agents of use for imaging and diagnosis of various diseased organs, tissues or cell types. The antibody may be labeled or conjugated with a fluorophore or radiotracer for use as an imaging agent. Many appropriate imaging agents are known in the art, as are methods for their attachment to proteins or peptides using metal chelate complexes, radioisotopes, fluorescent markers, or enzymes whose presence can be detected using a colorimetric markers (such as, but not limited to, urease, alkaline phosphatase, (horseradish) hydrogen peroxidase and glucose oxidase). In some embodiments, the imaging conjugate will also be dual labeled with a radio-isotope in order to combine imaging through nuclear approaches and be made into a unique cyclic structure and optimized for binding affinity and pharmacokinetics. Such agents can be administered by any number of methods known to those of ordinary skill in the art including, but not limited to, oral administration, inhalation, subcutaneous (sub-q), intravenous (I.V.), intraperitoneal (I.P.), intramuscular (I.M.), or intrathecal injection, or as described in greater detail below.

In some aspects, the imaging agent is a chromophore, such as a fluorophore. Exemplary fluorophores suitable for use with the present disclosure includes rhodamine, rhodol, fluorescein, thiofluorescein, aminofiuorescein, carboxyfiuorescein, chlorofluorescein, methylfluorescein, sulfofiuorescein, aminorhodol, carboxyrhodol, chlororhodol, methylrhodol, sulforhodol; aminorhodamine, carboxyrhodamine, chlororhodamine, methylrhodamine, sulforhodamine, and thiorhodamine; cyanine, indocarbocyanine, oxacarbocyanine, thiacarbocyanine, merocyanine, cyanine 2, cyanine 3, cyanine 3.5, cyanine 5, cyanine 5.5, cyanine 7, oxadiazole derivatives, pyridyloxazole, nitrobenzoxadiazole, benzoxadiazole, pyren derivatives, cascade blue, oxazine derivatives, Nile red, Nile blue, cresyl violet, oxazine 170, acridine derivatives, pro flavin, acridine orange, acridine yellow, arylmethine derivatives, auramine, crystal violet, malachite green, tetrapyrrole derivatives, porphin, phtalocyanine and bilirubin; 1-dimethylaminonaphthyl-5-sulfonate, 1-anilino-8-naphthalene sulfonate, 2-p-touidinyl-6-naphthalene sulfonate, 3-phenyl-7-isocyanatocoumarin, N-(p-(2-benzoxazolyl)phenyl)maleimide, stilbenes, pyrenes, 6-FAM (Fluorescein), 6-FAM (NHS Ester), Fluorescein dT, HEX, JOE (NHS Ester), MAX, TET, ROX, TAMRA, TARMA™ (NHS Ester), TEX 615, ATTO™ 488, ATTO™ 532, ATTO™ 550, ATTO™ 565, ATTO™ RholOl, ATTO™ 590, ATTO™ 633, ATTO™ 647N, TYE™ 563, TYE™ 665, and TYE™ 705. In particular aspects, the chromophore is TAMRA.

The detectable moiety may include, but is not limited to fluorodeoxyglucose (FDG); 2′-fluoro-2′deoxy-1beta-D-arabionofuranosyl-5-ethyl-uracil (FEAU); 5-[¹²³I]-2′-fluoro-5-iodo-1β-D-arabinofuranosyl-uracil; 5-[¹²⁴I]-2′-fluoro-5-iodo-1β-D-arabinofuranosyl-uracil; 5-[¹³¹I]-2′-fluoro-5-iodo-1β-D-arabinofuranosyl-uracil; 5-[¹⁸F]-2′-fluoro-5fluoro-1β-D-arabinofuranosyl-uracil; 2-[¹¹I]- and 5-([¹¹C]-methyl)-2′-fluoro-5 -methyl-1-β-D-arabinofuranosyl-uracil; 2-[¹¹C]-2′-fluoro-5-ethyl-1-β-D-arabinofuranosyl-uracil; 5-([¹¹C]-ethyl)-2′-fluoro-5-ethyl-1-β-D-arabinofuranosyl-uracil; 5-(2[¹⁸F]-ethyl)-2′-fluoro-5-(2-fluoro-ethyl)-1-β-D-arabinofuranosyl-uracil, 5-[¹²³I]-2′-fluoro-5-iodovinyl-1-β-D-arabinofuranosyl-uracil; 5-[¹²⁴I]-2′-fluoro-5-iodovinyl-1-β-D-arabinofuranosyl-uracil; 5-[¹³¹I]-2′-fluoro-5-iodovinyl-1-β-D-arabinofuranosyl-uracil; 5-[¹²³I]-2′-fluoro-5-iodo-1-β-D-ribofuranosyl-uracil; 5-[¹²⁴I]-2′-fluoro-5-iodo-1-β-D-ribofuranosyl-uracil; 5-[¹³¹I]-2′-fluoro-5-iodo-1-β-D-ribofuranosyl-uracil; 5[¹²³I]-2′-fluoro-5-iodovinyl-1-β-D-ribofuranosyl-uracil; 5-[¹²⁴I]-2′-fluoro-5-iodovinyl-1-β-D-ribofuranosyl-uracil; 5-[¹³¹I]-2′-fluoro-5-iodovinyl-1-β-D-ribofuranosyl-uracil; or 9-4-[¹⁸F]fluoro-3-(hydroxymethyl)butyl]guanine.

In some aspects, the imaging agent is a radionuclide. Suitable radionuclide labels are Tc, In, Ga, Cu, F, Lu, Y, Bi, Ac, and other radionuclide isotopes. Particularly, the radionuclide is selected from the group comprising ¹¹¹In, ^{99m}Tc, ^{97m}Tc, ⁶⁷Ga, ⁶⁶Ga, ⁶⁸Ga, ⁵²Fe, ⁶⁹Er, ⁷²As, ⁹⁷Ru, ²⁰³Pb, ⁶²Cu, ⁶⁴Cu, ⁶⁷Cu, ¹⁸⁶Re, ¹⁸⁸Re, ⁸⁶Y, ⁹⁰Y, ⁵¹Cr, ^{52m}Mn, ¹⁵⁷Gd, ¹⁷⁷Lu, ¹⁶¹Tb, ¹⁶⁹Yb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹⁷²Tm, ¹²¹Sn, ^{177m}Sn, ²¹³Bi, ¹⁴²Pr, ¹⁴³Pr, ¹⁹⁸Au, ¹⁹⁹Au, ¹⁸F, ¹²³I, ¹²⁴I, ¹³¹I, ⁷⁵Br, ⁷⁶Br, ⁷⁷Br, and ⁸²Br, amongst others. These radionuclides are cationic and can be complexed with the chelator through the chelating group of the conjugate to form labeled compositions.

Methods of detecting and/or for quantifying a detectable label or signal generating material depend on the nature of the label. The products of reactions catalyzed by appropriate enzymes can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers. Any of the methods for detection can be performed in any format that allows for any suitable preparation, processing, and analysis of the reactions. This can be, for example, in multi-well assay plates (e.g., 96 wells or 386 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label Imaging may be by optical imaging, ultrasound, PET, SPECT, MRI, or phototherapy.

In some aspects, the one or more assays may be sandwich ELISA assays. The three biomarkers may be detected by three separate ELISA assays, such as on three separate plates or slide for each biomarker or one plate or slide with separate wells for each biomarker.

In certain embodiments, the antigen-specific antibodies may be immobilized on a carrier or support (e.g., a bead, a magnetic particle, a latex particle, a microtiter plate well, a cuvette, or other reaction vessel). Examples of suitable carrier or support materials include agarose, cellulose, nitrocellulose, dextran, Sephadex®, Sepharose®, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, and the like. Binding agents may be indirectly immobilized using second binding agents specific for the first binding agents (e.g., mouse antibodies specific for the protein markers may be immobilized using sheep anti-mouse IgG Fc fragment specific antibody coated on the carrier or support).

In other aspects, the three biomarkers may be detected by a multiplex ELISA to detect two or three of the biomarkers simultaneously. For example, the multiplex ELISA may comprise an antibody array with capture antibodies spotted in subarrays on which the sample is incubated, non-specific proteins are washed off, and the array is incubated with a cocktail of biotinylated detection antibodies followed by a streptavidin-conjugated fluorophore which is visualized by a fluorescence laser scanner (e.g., Quantibody Multiplex ELISA Array, RayBiotech).

The presence of several different biomarkers in a test sample can be detected simultaneously using a multiplex assay, such as a multiplex ELISA. Multiplex assays offer the advantages of high throughput, a small volume of sample being required, and the ability to detect different proteins across a board dynamic range of concentrations. In certain embodiments, such methods employ an array, wherein multiple binding agents (for example, capture antibodies) specific for multiple biomarkers are immobilized on a substrate, such as a membrane, with each capture antibody being positioned at a specific, pre-determined, location on the substrate. Methods for performing assays employing such arrays include those described, for example, in US Patent Publication Nos. US2010/0093557A1 and US2010/0190656A1, the disclosures of which are hereby specifically incorporated by reference.

Multiplex arrays in several different formats based on the utilization of, for example, flow cytometry, chemiluminescence or electron-chemiluminesence technology, are well known in the art. Flow cytometric multiplex arrays, also known as bead-based multiplex arrays, include the Cytometric Bead Array (CBA) system from BD Biosciences (Bedford, Mass.) and multi-analyte profiling (xMAP®) technology from Luminex Corp. (Austin, Tex.), both of which employ bead sets which are distinguishable by flow cytometry. Each bead set is coated with a specific capture antibody. Fluorescence or streptavidin-labeled detection antibodies bind to specific capture antibody-biomarker complexes formed on the bead set. Multiple biomarkers can be recognized and measured by differences in the bead sets, with chromogenic or fluorogenic emissions being detected using flow cytometric analysis.

In an alternative format, a multiplex ELISA from Quansys Biosciences (Logan, UT) coats multiple specific capture antibodies at multiple spots (one antibody at one spot) in the same well on a 96-well microtiter plate. Chemiluminescence technology is then used to detect multiple biomarkers at the corresponding spots on the plate.

An antibody microarray may also be used to measure the differential expression of a plurality of biomarkers. For this, a plurality of antibodies is arrayed and covalently attached to the surface of the microarray or biochip. A protein extract containing the biomarker proteins of interest is generally labeled with a fluorescent dye or biotin. The labeled biomarker proteins are incubated with the antibody microarray. After washes to remove the unbound proteins, the microarray is scanned. The raw fluorescent intensity data may be converted into expression values using means known in the art.

### C. Imaging

In certain embodiments, this disclosure contemplates methods of imaging of target antigens using antibodies with detectable moieties. The antibody can be labeled with fluorescence and/or radioactivity which can be detected by various methods known in the art.

Nuclear Magnetic Resonance (NMR) and Magnetic Resonance Imaging (MRI) are techniques for identifying isotopes in a sample (area) by subjecting the sample to an external magnetic fields and detecting the resonance frequencies of the nuclei. An MRI scanner typically consists of magnet of 1.5 to 7, or more Tesla strength. A magnetic field and radio waves are used to excite protons in the body. These protons relax after excitation, and a computer program translates this data into pictures of human tissue. In certain embodiments, this disclosure contemplates that a pre-contrast image is taken. Once the composition is injected, a post-contrast image is taken.

NMR typically involves the steps of alignment (polarization) of the magnetic nuclear spins in an applied, constant magnetic field and perturbation of this alignment of the nuclear spins by employing an electro-magnetic radiation, usually radio frequency (RF) pulse. A pulse of a given carrier frequency contains a range of frequencies centered about the carrier frequency. The Fourier transform of an approximately square wave contains contributions from the frequencies in the neighborhood of the principal frequency. The range of the NMR frequencies allows one to use millisecond to microsecond radio frequency pulses.

Single-photon emission computed tomography (SPECT) is an imaging technique using gamma rays. Using a gamma camera, detection information is typically presented as cross-sectional slices and can be reformatted or manipulated as required. One injects a gamma-emitting radioisotope (radionuclide) into a subject. The radioisotope contains or is conjugated to a molecule that has desirable properties, e.g., a marker radioisotope has been attached to a ligand, folate. This allows the combination of ligand, e.g., folate, and radioisotope (the radiopharmaceutical) to be carried and bound to a place of interest in the body, which then (due to the gamma-emission of the isotope) allows the ligand concentration to be seen by a gamma-camera.

Positron emission tomography (PET) is an imaging technique that produces a three-dimensional image. The system detects pairs of gamma rays emitted indirectly by a positron-emitting radionuclide (tracer). Three-dimensional images of tracer concentration within the area are then constructed by computer analysis. A radioactive tracer isotope is injected into subject, e.g., into blood circulation. Typically there is a waiting period while tracer becomes concentrated in tissues of interest; then the subject is placed in the imaging scanner. As the radioisotope undergoes positron emission decay, it emits a positron, an antiparticle of the electron with opposite charge, until it decelerates to a point where it can interact with an electron, producing a pair of (gamma) photons moving in approximately opposite directions. These are detected in the scanning device. The technique depends on simultaneous or coincident detection of the pair of photons moving in approximately opposite direction (the scanner has a built-in slight direction-error tolerance). Photons that do not arrive in pairs (i.e. within a timing-window) are ignored. One localizes the source of the photons along a straight line of coincidence (also called the line of response, or LOR). This data is used to generate an image.

Light having a wavelength range from 600 nm and 850 nm lies within the near infrared range of the spectrum, in contrast to visible light, which lies within the range from about 400 nm to about 500 nm. Therefore, the excitation light used in practice of the disclosure diagnostic methods will contain at least one wavelength of light to illuminates the tissue at the infrared wavelength to excite the compounds in order that the fluorescence obtained from the area having uptake of the compounds of the present disclosure is clearly visible and distinct from the auto-fluorescence of the surrounding tissue. The excitation light may be monochromatic or polychromatic. In this manner, the compounds of the present disclosure are advantageous as they eliminate the need for use of filtering mechanisms that would be used to obtain a desired diagnostic image if the fluorescent probe is one that fluoresces at wavelengths below about 600 nm. In this manner, the compounds of the present disclosure avoid obscured diagnostic images that are produced as a result of excitation light of wavelengths that would be reflected from healthy tissue and cause loss of resolution of the fluorescent image.

Diagnostic labs, physicians' offices and operating rooms for surgical procedures can be equipped with an overhead light that produces wavelengths of light in the optical emitting spectrum useful in practice of disclosure diagnostic methods, such as lamps that produce light in the appropriate wavelength. Such a light can be utilized in the practice of the disclosure diagnostic methods merely by turning out the other lights in the operating room (to eliminate extraneous light that would be visibly reflected from tissue in the body part under investigation) and shining the excitation light of near infrared wavelength into the body cavity or surgically created opening so that the fluorescent image received directly by the eye of the observer (e.g., the surgeon) is predominantly the fluorescent image emanating from the fluorophore(s) in the field of vision.

Within any of the imaging embodiments, methods disclosed herein may further comprise the steps of recording the images from an area of the subject on a computer or computer readable medium. In certain embodiments, the methods may further comprise transferring the recorded images to a medical professional representing the subject under evaluation.

In some aspects, the compounds of the present disclosure are used to identify a tumor by administering such compounds for a time and under conditions that allow for binding of the compound to at least one cell of the target cell type (e.g., recently recruited and differentiated macrophages). The bound compound is then optically detected such that presence of fluorescence of the near infrared wavelength emanating from the bound, targeted compound of the present disclosure indicated that the target cell type is present in the biological sample.

The amount of the conjugate compound effective for use in accordance with the method of the disclosure depends on many parameters, including the molecular weight of the conjugate, its route of administration, and its tissue distribution. The antigen-specific antibodies can be administered in one or more doses (e.g., about 1 to about 3 doses) prior to the catheterization or external imaging procedure. The number of doses depends on the molecular weight of the compound, its route of administration, and its tissue distribution, among other factors.

The antibodies may be administered parenterally to the patient being evaluated for a tumor, for example, intravenously, intradermally, subcutaneously, intramuscularly, or intraperitoneally, in combination with a pharmaceutically acceptable carrier. Suitable means for parenteral administration include needle (including microneedle) injectors, needle-free injectors and infusion techniques.

### III. METHODS OF USE

Aspects of the present disclosure include methods for diagnosing or monitoring the onset, progression, or regression of a disease in a subject by, for example, obtaining samples from a subject and assaying such samples for the presence and/or expression of a target biomarker.

Certain embodiments of the present methods and compositions have applicability in high sensitivity (pg/mL) quantification of protein biomarkers in biofluid samples, including blood, serum, saliva, urine, etc. Current state of the art technology for protein quantification requires an ELISA test or bead-based assays (SIMOA) which are expensive, laborious, time-consuming and need to be performed in a laboratory setting. Certain embodiments of the present methods can achieve similar sensitivity as ELISA, while being much simpler to perform, and at least 3 times faster, without requiring a laboratory, making it well suited for rapid disease detection and screening at point-of-care settings. Additionally, this technology can be readily modified for multiplexed measurements of multiple biomarkers and/or multiple samples by using a multi-channel potentiostat.

Certain embodiments of the present methods may be adapted for use with whole blood samples. Further, the present methods may be adapted for the detection of other biomarkers associated with other diseases, such as HIV and cancer.

In some embodiments, the target biomarker is typically selected from viral infectious diseases such as influenza, preferably influenza-A, influenza-B, influenza-C or thogotovirus, more preferably influenza-A comprising e.g., haemagglutinin subtypes H1, H2, H3, H4, H5, H6, H7, H8, H9, H10, H11, H12, H13, H14 or H15, and/or neuroamidase subtypes N1, N2, N3, N4, N5, N6, N7, N8 or N9, or preferably influenza-A subtypes H1N1, H1N2, H2N2, H2N3, H3N1, H3N2, H3N3, H5N1, H5N2, H7N7 or H9N2, etc., or any further combination, malaria, severe acute respiratory syndrome (SARS), respiratory syncytial virus infection, yellow fever, AIDS, Lyme borreliosis, Leishmaniasis, anthrax, meningitis, Condyloma acuminata, hollow warts, Dengue fever, three-day fever, Ebola virus, cold, early summer meningoencephalitis (FSME), shingles, hepatitis, herpes simplex type I, herpes simplex type II, Herpes zoster, Japanese encephalitis, Arenavirus-associated diseases (Lassa fever infection), Marburg virus, measles, foot-and-mouth disease, mononucleosis infectiosa (Pfeiffer's glandular fever), mumps, Norwalk virus infection, smallpox, polio (childhood lameness), pseudo-croup, Erythema infectiosum (fifth disease), rabies, warts, West Nile fever, chickenpox, Cytomegalovirus (CMV); bacterial infectious diseases such as prostate inflammation, anthrax, appendicitis, borreliosis, botulism, Camphylobacter, Chlamydia trachomatis (inflammation of the urethra, conjunctivitis), cholera, diphtheria, donavanosis, epiglottitis, typhus fever, gas gangrene, gonorrhoea, rabbit fever, Heliobacter pylori, whooping cough, climatic bubo, osteomyelitis, Legionnaire's disease, leprosy, listeriosis, pneumonia, meningitis, bacterial meningitis, anthrax, otitis media, Mycoplasma hominis, neonatal sepsis (Chorioamnionitis), noma, paratyphus, plague, Reiter's syndrome, Rocky Mountain spotted fever, Paratyphoid fever, Typhoid fever, scarlet fever, syphilis, tetanus, tripper, tsutsugamushi disease, tuberculosis, typhus, vaginitis (colpitis), soft chancre; and infectious diseases caused by parasites, protozoa or fungi, such as amoebiasis, bilharziosis, Chagas disease, Echinococcus, fish tapeworm, fish poisoning (Ciguatera), fox tapeworm, athlete's foot, canine tapeworm, candidosis, yeast fungus spots, scabies, cutaneous Leishmaniosis, lambliasis (giardiasis), lice, malaria, onchocercosis (river blindness), fungal diseases, bovine tapeworm, schistosomiasis, porcine tapeworm, toxoplasmosis, trichomoniasis, trypanosomiasis (sleeping sickness), visceral Leishmaniosis, nappy/diaper dermatitis or miniature tapeworm.

In some aspects, the target biomarker is selected from Influenza A virus, influenza B virus, respiratory syncytial virus, parainfluenza virus, Streptococcus pneumoniae, Corynebacterium diphtheriae, Clostridium tetani, Measles, Mumps, Rubella, Rabies virus, Staphylococcus aureus, Clostridium difficile, Mycobacterium tuberculosis, Candida albicans, Haemophilus influenzae B (HiB), poliovirus, hepatitis B virus, human papillomavirus (HPV), human immunodeficiency virus, SARS CoV, Pertussis toxin, polio virus, Plasmodium, Staphylococcus aureus, Bordetella pertussis, and/or polio virus VP1-4. In particular aspects, the viral pathogenic target nucleic acids are specific to human immunodeficiency virus (HIV), herpes simplex virus (HSV-1), Influenza A virus, West Nile Virus, and/or Epstein-Barr virus (EBV) viral pathogen nucleic acids.

### IV. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Examples 1—Material and Methods

Biochemicals and Reagents. Dimethyl sulfoxide (DMSO), phosphate-buffered saline (PBS, pH 7.4), (ethylenedinitrilo)-tetraacetic acid (EDTA), 2-Iminothiolane hydrochloride, human serum (from male AB-clotted whole blood), and 3,3′,5,5′-tetramethylbenzidine (TMB) substrate (supersensitive) were purchased from Sigma-Aldrich (St Louis, MO). N-(3-Dimethylaminopropyl)-N′-ethylcarbodiimide (EDC) and N-hydroxysuccinimide (NHS) were obtained from Thermo Fisher Scientific (Waltham, MA). StabilBlock immunoassay stabilizer, StabilCoat Plus immunoassay stabilizer, StabilZyme HRP stabilizer, and MatrixGuard assay diluent were purchased from SurModics, Inc. (Eden Prairie, MN). Carboxylated magnetic nanobeads (200 nm) were purchased from Ademtech (Pessac, France). SARS-CoV-2 nucleocapsid protein was obtained from Advaite, Inc. (Malvern, PA). Mouse monoclonal SARSCoV/SARS-CoV-2 nucleocapsid antibody [6H3] (GTX632269), rabbit polyclonal SARS-CoV-2 nucleocapsid antibody (GTX135357), SARS-CoV-2 nucleocapsid antibody pair [HL5410/HL455-MS] (GTX500042), and horseradish peroxidase (HRP)-conjugated rabbit monoclonal SARS-CoV-2 nucleocapsid antibody [HL448] (GTX635686-01) were purchased from GeneTex (Irvine, CA). Human monoclonal anti-SARS-CoV-2 nucleocapsid antibody [SQab20177] (ARG66735), MERS-CoV nucleocapsid recombinant protein (His-SUMO tagged, N-ter), and SARS-CoV nucleocapsid recombinant protein (His-SUMO tagged, N-ter) were purchased from Arigo (Taiwan, ROC). Recombinant SARS-CoV-2 spike glycoprotein RBD (ab273065) was obtained from Abcam (Cambridge, MA). Deidentified serum samples obtained from healthy volunteers and COVID-19 patients were purchased from BioIVT (NY, USA).

Preparation of Dually-Labeled Magnetic Nanobeads. DMBs were prepared by dispersing 1 mg of carboxylated magnetic nanobeads in 400 μL of MES buffer (pH 5.0, 25 mM) and washing thrice (gentle agitation for 5 min followed by magnetic separation for 5 min and subsequent removal of the supernatant). Next, 100 μL of MES buffer containing HRP and detection antibody (dAb) at a 400:1 molar ratio was mixed with the nanobeads preactivated with 10 mg/mL of EDC/NHS and incubated overnight at room temperature. After washing with PBS and blocking of nonspecific binding sites with a StabilCoat Plus stabilizer, the DMBs were dispersed in 400 μL of StabilZyme HRP stabilizer to a final concentration of 2.5 mg/mL and used immediately or stored at 4° C. for up to 2 weeks.

Preparation of Immunosensors. Screen-printed gold electrode (SPGE) sensors were obtained from Metrohm AG (Herisau, Switzerland). Capture antibodies (cAbs) were first thiolated by incubating 100 μL of cAb at 50 μg/mL with 100-fold molar excess of 2-iminothiolane in PBS containing 2 mM of EDTA for 1 hour at room temperature, followed by centrifugation for 25 min at 13,800 g to remove excess reagents. Thiolated cAbs were immobilized on the SPGE sensor by incubating 6 μL of cAb solution at 50 μg/mL on the working electrode (WE) for 2 h at room temperature, followed by rinsing with PBS and gently drying with purified N2. StabilBlock stabilizer solution was dispensed on the sensor and dried at room temperature to passivate the surface and enhance the stability of the immobilized cAb. Sensors were stored at room temperature in a desiccator (<15% RH) and used within 1 week. Fabrication of Microfluidic Chips. The microfluidic chips consist of a 100 μm-thick polyethylene terephthalate (PET) film (McMaster-Carr) stacked with a 3 mm-thick poly(methyl methacrylate) (PMMA) cartridge on top of an immunosensor. Microchannels and microfluidic components were designed using AutoCAD software (Autodesk, Inc.). Microchannels, inlets, and outlets were generated in the PET and PMMA layers using a CO2 laser cutter (Universal Laser Systems, Scottsdale, AZ). The PET film, PMMA cartridge, and SPGE sensor were bonded together using double-sided adhesive film (Adhesives Research, PA).

Electrochemical Measurements. Electrochemical measurements were performed at ambient conditions using either a PalmSens4 potentiostat connected to a desktop PC or a Sensit Smart potentiostat connected to a Google Pixel 2 smartphone. Prior to measurements, 2.5 μL of DMB solution was mixed with 50 μL of serum spiked with N protein or clinical serum specimens, vortexed for 5 s, and dispensed into the microfluidic chip. Spiked serum samples were either used as is or diluted 5× in MatrixGuard assay diluent. For measurements using the PalmSens4 and desktop PC, the sample was infused through the chip for 30 seconds at 100 μL/min using a syringe pump (KD Scientific, MA). For measurements using the smartphone-based sensing device, the sample was dispensed into the chip using a capillary tube and plunger (Abbott). The microfluidic chip was then placed on a 4 mm neodymium magnet (McMaster-Carr) for 1 minute to concentrate the DMBs on the WE and incubated in the dark for either 50 minutes for whole serum samples or 25 minutes for diluted serum samples. Measurements of clinical serum specimens were performed by diluting samples 5× in an assay diluent (to conserve the sample for replicate measurements), followed by immunomagnetic enrichment and incubation for 25 minutes. A wash buffer (1×PBS containing 0.05% Tween-20) was flushed through the chip for 4 minutes at 100 μL/min, followed by a TMB substrate for 1 minute at 100 μL/min for measurements using the PalmSens4 and desktop PC. For measurements using the smartphone-based sensing device, a 1 cc plastic syringe (Thermo Fisher Scientific) was inserted into the inlet of the microfluidic chip and used to purge the sample from the chip, followed by the sequential application of 80 μL of wash buffer and 80 μL of TMB substrate into the chip using fresh capillary tubes and plungers. After 2 min, chronoamperometric measurements were performed by applying a bias potential of −0.2 V (vs Ag/AgCl) for 100 s. Current values were averaged over the final 5 s of chronoamperograms.

### Example 2—Design and Characterization Microfluidic Assay

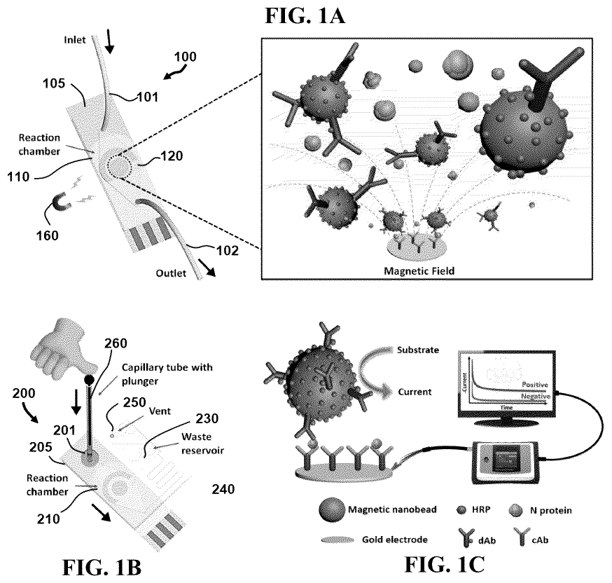

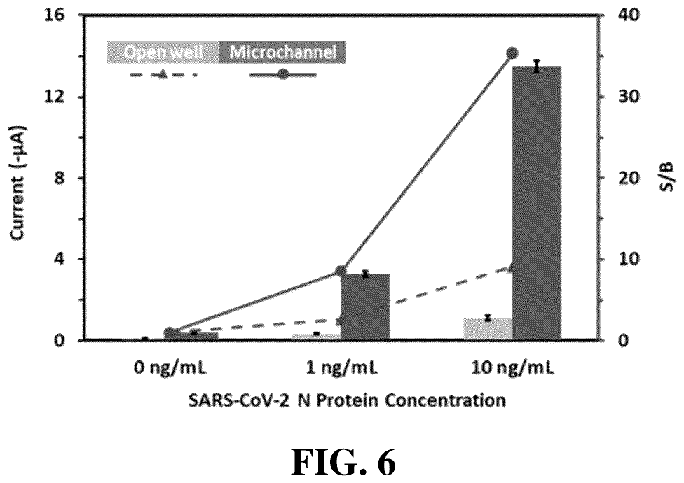

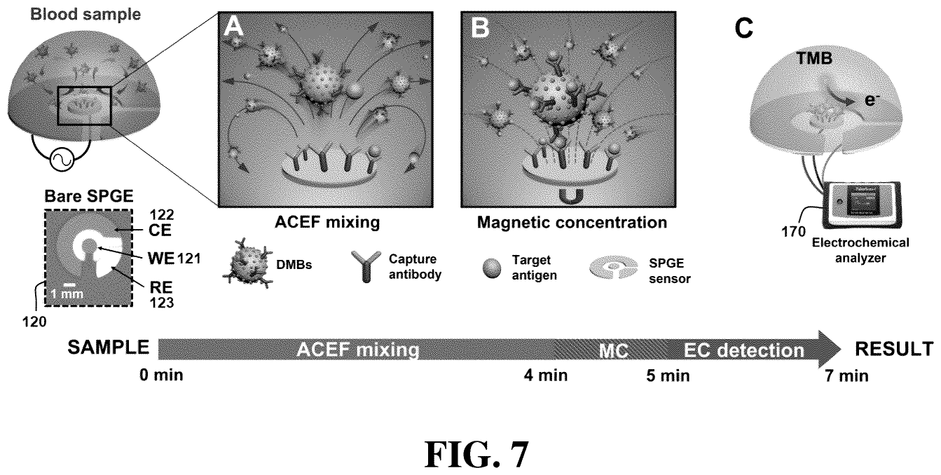

Design of the Microfluidic Chip. The integration of this immunosensor on a microfluidic platform offers several advantages over open well format immunoassays. Specifically, the recommended working volume for a standard 96-well microtiter plate is 100-200 μL, whereas the microfluidic immunosensor requires only 25 μL of sample and 80 μL of reagent per measurement. In addition, sample processing and liquid handling for open well format assays involve multiple pipetting steps, which are tedious and time-consuming. In contrast, sample processing (immunomagnetic enrichment) and liquid handling (sensor washing) are performed directly on the microfluidic chip, which minimizes the labor and time required for each measurement, facilitating its use for point-of care testing. Lastly, the integration of immunosensors with microfluidics has been shown to significantly reduce the time for antibody-antigen reactions and enhance the detection sensitivity compared with open well format immunoassays. Ng, A. H. C.; Uddayasankar, U.; Wheeler, A R Immunoassays in Microfluidic Systems. Analytical and Bioanalytical Chemistry; Springer Jun. 27, 2010, pp 991-1007; Choi, C. J.; Belobraydich, A. R.; Chan, L. L.; Mathias, P. C.; Cunningham, B. T. Comparison of Label-Free Biosensing in Microplate, Microfluidic, and Spot-Based Affinity Capture Assays. Anal. Biochem. 2010, 145, 1. The analytical performance of the microfluidic immunosensor was briefly compared with an open-well immunosensor and it was observed that the amperometric currents and signal-to-background (S/B) ratios generated from the microfluidic immunosensor were 3-4× higher than those generated from the open-well immunosensor (FIG. 6). Different microfluidic chips were designed for measurements using the PalmSens4-based sensing platform and the smartphone-based diagnostic device. For measurements using the PalmSens4, an apparatus 100 comprises a microfluidic chip 105 comprising a reaction chamber 110 as shown in FIG. 1A. In the embodiment shown, reaction chamber 110 is configured as a 400 μm-high reaction chamber encompassing an immunosensor 120 coupled to an inlet 101 and an outlet 102. Additional details of immunosensor 120 are provided below in the discussion of FIG. 7.

As shown in FIG. 1B, an apparatus 200 comprises a microfluidic chip 205 for measurements using the Sensit Smart and smartphone comprises a reaction chamber 210 encompassing an immunosensor 220 connected to a waste reservoir 230 via a serpentine channel 240 and an air vent 250. In particular embodiments, serpentine channel 240 is 500 μm-wide, reaction chamber 210 is 400 μm-high and waste reservoir 230 is 9×12 mm. In certain embodiments, a rubber gasket is installed at the inlet of the chip to facilitate the insertion of the capillary tube and prevent leaking. The embodiment shown in FIG. 1B also comprises a sample loading mechanism 260 (e.g. a capillary tube with a plunger in this embodiment) coupled to an inlet 201.

Design of the Electrochemical Magneto Immunoassay. Prior works have demonstrated the use of antibody-labeled magnetic beads for immunomagnetic enrichment and signal amplification, enabling sensitive analyte detection in complex biofluids. See, MM, J., et al., “Integrated Biosensor for Rapid and Point-of-Care Sepsis Diagnosis,” ACS Nano 2018, 12, 3378-3384; Valverde, A., “Electrochemical Immunoplatform to Improve the Reliability of Breast Cancer Diagnosis through the Simultaneous Determination of RANKL and TNF in Serum.” Sens. Actuators, B 2020, 314, 128096. Otiena et al. reported a microfluidic magneto immunoassay for multiplexed detection of a parathyroid hormone-related peptide and peptide fragments in serum. Otieno, B. A., “Cancer Diagnostics via Ultrasensitive Multiplexed Detection of Parathyroid Hormone-Related Peptides with a Microfluidic Immunoarray,” Anal. Chem. 2016, 88, 9269-9275. While this assay was capable of performing ultrasensitive protein measurements, the experimental setup involves multiple components (e.g., magnetic stirrer, sample injector, syringe pump, switching valve, etc.), hindering its use for point-of-care applications. In this embodiment, a simple and rapid (1 min) method was used for immunomagnetic enrichment using a low-cost neodymium magnet 160 proximal to immunosensor 150 as shown in FIG. 1A. The serum sample is premixed with DMBs prior to loading into the microfluidic chip, which is carried out using either a syringe pump or capillary tubes and plungers (for the smartphone-based device). If the sample contains the target antigen, it binds to the DMB and forms a DMB-antigen immunocomplex. When the chip is placed on the magnet, a magnetic field is generated, causing the DMB-antigen immunocomplexes to rapidly migrate to the sensor surface where they subsequently bind to the cAb-immobilized WE (FIG. 1A). In the presence of the TMB substrate, the HRP-coated DMBs catalyze the reduction of TMB upon application of a bias potential, which generates an amperometric current that is proportional to the concentration of target antigen attached to the sensor surface (FIG. 1C). If the sample does not contain the target antigen, then the DMBs are washed away from the sensor surface and a negligible electrochemical signal is generated upon the application of a bias potential.

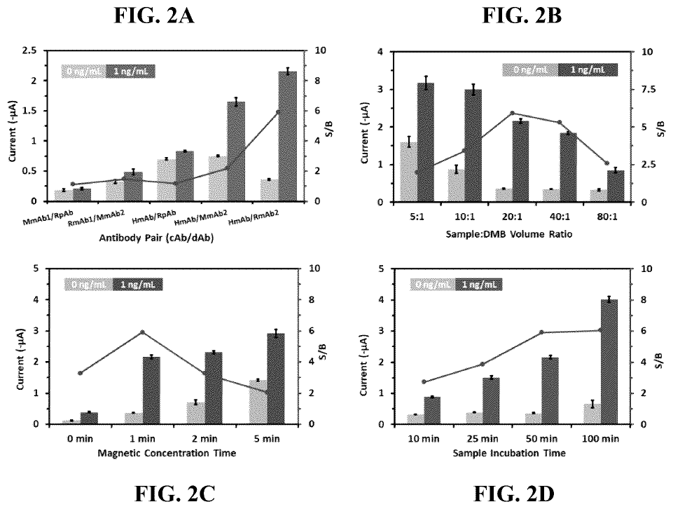

Optimization of Assay Parameters. Several assay parameters, including the antibody pair, sample to DMB solution volume ratio, magnetic enrichment time, and incubation time, were optimized to enhance the analytical performance of this immunosensor for SARS-CoV-2 N protein detection. One of the most important parameters that affects the performance of immunoassays is the antibody affinity toward the target antigen. There are numerous SARS-CoV-2 N protein antibodies that are commercially available, and each one possesses a specific antigenicity to the SARS-CoV-2 N protein. Therefore, to determine the optimal antibody pair for the immunosensor, measurements of SARSCoV-2 N protein spiked in whole serum at 0 and 1 ng/mL were performed using SPGE sensors with five different antibody pairs. The cAbs were immobilized on the WE of the sensors as described in “Preparation of Immunosensors,” and dAbs were conjugated with DMBs as described in “Preparation of Dually-Labeled Magnetic Nanobeads”. The amperometric signals generated using the five antibody pairs are presented in FIG. 2A. Antibody pairs consisting of a mouse or rabbit cAb generated very low amperometric signals (<0.5 μA) and low S/B ratios of <2, indicating poor antigenicity to SARS-CoV-2 N protein because they are raised against nonhuman species. Amperometric signals generated from immunosensors using a human monoclonal cAb were significantly larger than those generated from sensors using a nonhuman monoclonal cAb; however, when paired with a mouse monoclonal antibody or rabbit polyclonal antibody as the dAb, a very high background signal was observed, resulting in negligible improvement in the S/B ratio. Lastly, the use of a rabbit monoclonal antibody conjugated with HRP was evaluated as the dAb, which generated a large electrochemical current with a low background signal, resulting in a S/B ratio of ˜6. Thus, a human monoclonal cAb and an HRP-conjugated rabbit monoclonal dAb were selected as the optimal antibody pair and used for subsequent assay optimization experiments. The sample to DMB solution ratio was optimized by performing measurements of serum samples spiked with increasing concentrations of SARS-CoV-2 N protein using varying volumes of DMB solution. As shown in FIG. 2B, the amperometric signal is correlated with the sample/DMB volume ratio where measurements using higher sample/DMB volume ratios resulted in lower electrochemical currents. However, measurements using low sample/DMB volume ratios (<10:1) resulted in high background signals and low S/B ratios (<3.5) due to an excessive amount of DMBs, which increases the likelihood of nonspecific binding of DMBs on the sensor. As the sample/DMB volume ratio increases, the background signal decreases until a sample/DMB volume ratio of 20:1, after which point, the background signal remains constant. The largest S/B ratio (˜5.5) was obtained using a sample/DMB volume ratio of 20:1, which was selected as the optimal volume ratio. Experiments were also performed to optimize the magnetic enrichment time by detecting SARS-CoV-2 N protein spiked in serum samples at 0 ng/mL and 1 ng/mL with varying durations of magnetic enrichment (FIG. 2C). With no magnetic enrichment, a very low (<0.5 μA) amperometric signal was generated at 1 ng/mL, resulting in a S/B ratio of ˜3. Applying magnetic concentration for 1 min resulted in a significant increase in the amperometric signal by 5×, compared with no magnetic enrichment, with a minimal rise in the background signal (S/B ratio of ˜6). These results demonstrate that the migration of DMBs to the sensor surface is significantly enhanced in the presence of a magnetic field, which facilitates the attachment of antigen-DMB immunocomplexes on the cAb-coated immunosensor. Applying magnetic concentration for >1 min resulted in a minimal rise in the amperometric signal with a more pronounced increase in the background signal, causing the S/B ratio to decrease. It was hypothesized that the increase in the background signal with longer magnetic enrichment durations (>1 min) is due to the accumulation and subsequent trapping of unbound DMBs on the coarse SPGE sensor surface, which cannot be completely removed with laminar flow rinsing. The last parameter that was studied was the post immunomagnetic enrichment incubation time. Measurements were performed using serum samples spiked with SARS-CoV-2 N protein at 0 and 1 ng/mL using a magnetic concentration duration of 1 min with varying incubation times. As shown in FIG. 2D, longer incubation times resulted in higher S/B ratios until a steady state was reached at 50 min. While larger amperometric signals can be generated with incubation times longer than 50 min, the background signal also increases proportionally, leading to a negligible improvement in the S/B ratio. Therefore, 50 min was selected as the optimal incubation time.

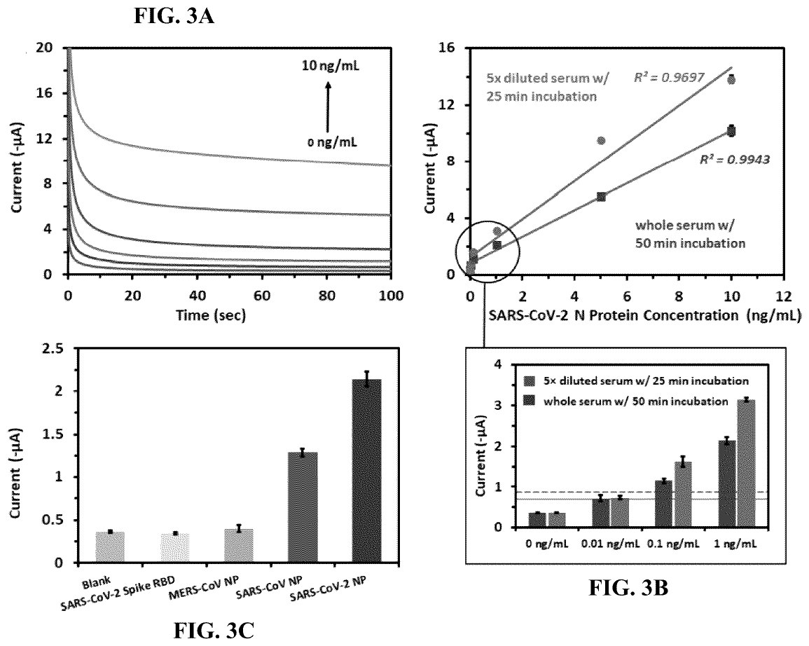

Detection of SARS-CoV-2 N Protein in Serum. Measurements of whole serum and 5× diluted serum spiked with increasing concentrations of SARS-CoV-2 N protein were carried out to assess the analytical performance of this immunosensor. Chronoamperograms generated from whole serum samples containing SARS-CoV-2 N protein from 0 to 10 ng/mL are shown in FIG. 3A, which show a positive correlation between the amperometric current and SARS-CoV-2 N protein concentration. Calibration plots based on amperometric currents at 100 s for whole serum and 5× diluted serum are presented in FIG. 3B. The response of this sensor is highly linear for whole serum with a R2 correlation coefficient of 0.9943. The linearity of the calibration curve for 5× diluted serum (R2=0.9697) is lower than that for whole serum, which is likely due to the use of a supersensitive TMB substrate, resulting in limited reaction kinetics at higher (>1 ng/mL) analyte concentrations. While the use of an alternative TMB substrate could improve the linearity, this could lead to a less desirable analytical performance with a lower detection sensitivity. The lower LOD, calculated as 3× the SD at 0 ng/mL divided by the slope of the calibration curve, of this immunosensor for SARS-CoV-2 N protein detection in whole serum and 5× diluted serum is 50 and 10 pg/mL, respectively. the improved sensitivity obtained from diluted serum compared with whole serum was attributed to the use of a commercial assay diluent, which contains blocking agents that inhibit/neutralize the interference of antigen-antibody binding caused by endogenous components, such as heterophilic antibodies and human anti-animal antibodies, in the sample matrix. Tate, J.; Ward, G. Interferences in Immunoassay. Clin. Biochem. Rev. 2004, 25, 105-120; Kricka, L. J. Human Anti-Animal Antibody Interferences in Immunological Assays. Clin. Chem. 1999, 45, 942-956; Spengler, M.; Adler, M.; Niemeyer, C. M. Highly Sensitive Ligand-Binding Assays in Pre-Clinical and Clinical Applications: Immuno-PCR and Other Emerging Techniques. Analyst 2015, 140, 6175-6194. The results were consistent with those reported in prior works, which demonstrate that matrix interference effects in immunoassays can be diminished by using heterophilic antibody blocking agents. See, DeForge, L. E., “Evaluation of Heterophilic Antibody Blocking Agents in Reducing False Positive Interference in Immunoassays for IL-17AA, IL-17FF, and IL-17AF. J,” Immunol. Methods 2010, 362, 70-81; Nicholson, S.; Fox, M.; Epenetos, A.; Rustin, G. Immunoglobulin Inhibiting Reagent: Evaluation of a New Method for Eliminating Spurious Elevations in CA125 Caused by HAMA. Int. J. Biol. Markers 1996, 11, 46-49. While a lower LOD can be achieved using 5× diluted serum with a shorter 25 min incubation time, this requires the serum sample to be diluted prior to the measurement. For applications where sample dilution is undesired, whole serum samples can be used requiring a slightly longer (50 min) incubation time to achieve high sensitivity detection. The sensitivity of this immunosensor is within the range of SARS-CoV-2 N protein serum levels in individuals infected with COVID-19 (1 pg to >10,000 pg/mL), suggesting that it will be suitable as a diagnostic tool for the detection of COVID-19 infection. See, Shan, D., “SARS-Coronavirus-2 nucleocapsid protein measured in blood using a Simoa ultra-sensitive immunoassay differentiates COVID-19 infection with high clinical sensitivity,” 2020, medRxiv: 2020.08.14.20175356. The specificity of this immunosensor was evaluated by performing measurements of whole serum samples spiked with 1 ng/mL of SARS-CoV-2 Spike RBD, another biomarker of COVID-19 infection, SARS-CoV N protein, MERS-CoV N protein, and nonspiked serum. As shown in FIG. 3C, the amperometric signals generated from the samples containing SARS-CoV-2 Spike RBD and MERS-CoV N protein are similar to the nonspiked serum sample (blank control), indicating that these protein biomarkers do not cross-react with this immunosensor. The amperometric signal from the sample containing SARS-CoV N protein is ˜1.5× larger than the background signal, indicating moderate cross-reactivity with the SARS-CoV-2 N protein antibody used in this assay. This is due to >90% conserved similarity in protein sequences between SARS-CoV-2 and SARS-CoV. See, Zeng, W., “Biochemical Characterization of SARSCoV-2 Nucleocapsid Protein,” Biochem. Biophys. Res. Commun. 2020, 527, 618-623. While cross-reactivity between SARS-CoV N protein and SARS-CoV-2 N antibodies has been previously reported and is an issue for all immunoassays utilizing SARS-CoV-2 N protein antibodies, its impact on the current COVID-19 pandemic is negligible because the number of individuals infected with SARS-CoV is very small compared with SARS-CoV-2 and no new SARSCoV outbreaks have been reported for nearly two decades. Shrock, E., et a., “Viral Epitope Profiling of COVID-19 Patients Reveals Cross-Reactivity and Correlates of Severity,” Science 2020, 370, No. eabd4250; Ma, Z.; Li, P.; Ji, Y.; Ikram, A.; Pan, Q., “Cross-Reactivity towards SARS-CoV-2: The Potential Role of Low-Pathogenic Human Coronaviruses,” Lancet Microbe 2020, 1, No. e151.

### Example 3—SARS-CoV-2 N Protein Detection Using a Smartphone

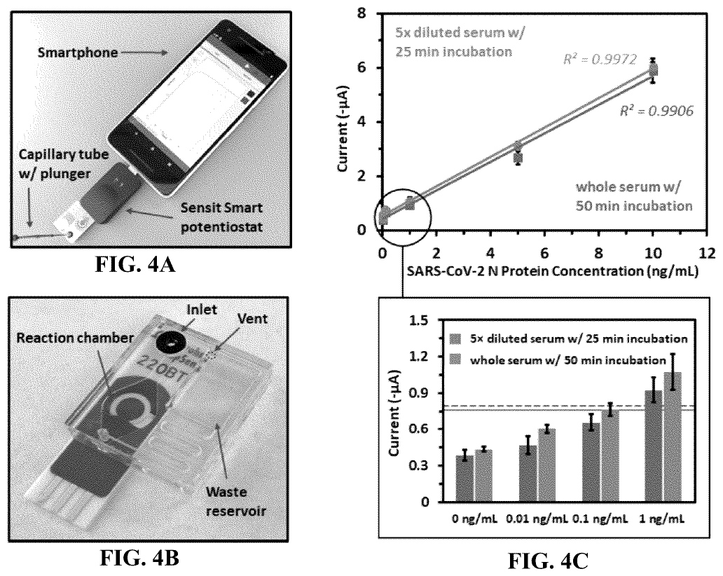

To enhance the portability and simplicity of this immunosensor, a handheld diagnostic device was also developed for quantitative measurements of SARS-CoV-2 N protein in serum. As shown in FIG. 4A, this device consists of a Google Pixel 2 smartphone, Sensit Smart potentiostat, and microfluidic immunosensor chip. The microfluidic chip incorporates a waste reservoir to store the liquid samples after being dispensed into the chip (FIG. 4B). The sample, wash buffer, and TMB substrate are sequentially dispensed into the chip using capillary tubes and plungers, which circumvents the need for an external pump and power source. It was observed that the washing effectiveness using a capillary tube and plunger is lower than that using a syringe pump, which can diminish the detection sensitivity and/or sensor reproducibility. Therefore, an additional step was added to purge the microchamber with air using a 1 cc plastic syringe after each liquid loading step to enhance the removal of unbound DMBs and nonspecific species from the sensor. To evaluate the analytical performance of this device, electrochemical measurements were performed using whole serum and 5× diluted serum samples spiked with increasing concentrations of SARSCoV-2 N protein. Calibration plots for whole serum and 5× diluted serum samples are presented in FIG. 4C, which exhibit excellent linearity with R2 correlation coefficients of 0.9906 and 0.9972, respectively. The lower LOD calculated for whole serum and 5× diluted serum samples is 230 pg/mL and 100 pg/mL, respectively. The detection sensitivity obtained using the smartphone-based device is lower than that using the PalmSens4-based sensing platform because of the reduced effectiveness of the capillary tube and plunger to fully rinse the sensor surface. However, the sensitivity of the handheld device is much higher compared with rapid COVID-19 antigen tests, while offering similar portability, simplicity, and speed, making it useful for point-of-care testing.

### Example 4—SARS-CoV-2 N Protein Detection in Clinical Serum Specimens

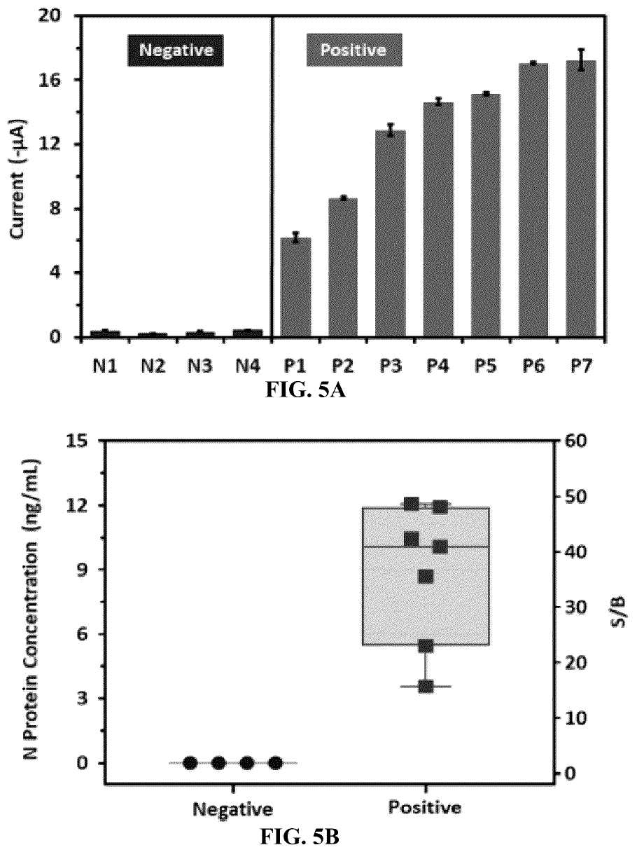

To evaluate the utility of this immunosensor for diagnosing COVID-19 infection, measurements were performed using serum samples obtained from COVID-19 patients confirmed by RT-PCR (P1-P7) and from healthy, uninfected individuals (N1-N4). Samples N1-N3 were collected pre-COVID-19 from healthy volunteers and sample N4 was obtained from an individual with a negative PCR COVID-19 test result. As shown in FIG. 5A, the electrochemical signals generated from specimens obtained from uninfected individuals (N1-N4) are very low (<1 μA). In contrast, the electrochemical signals generated from the specimens obtained from COVID-19 patients are at least 5× larger, ranging from ˜5 to 17 μA, which is consistent with the PCR results. Using the calibration plot in FIG. 3B, the calculated SARS-CoV-2 N protein concentration and corresponding S/B ratios were determined for the clinical specimens. The data was normalized so that the lowest calculated N protein concentration (which was a negative value) was set to 0 ng/mL (and 1 for the S/B ratio). As shown in FIG. 5B, the calculated levels of SARS-CoV-2 N protein in COVID-19 positive specimens range from ˜3 to 12 ng/mL, which is consistent with those measured by Torrente-Rodriǵuez et al. using a graphene-based immunosensor.17

Based on these preliminary results, this immunosensor can accurately distinguish COVID-19 patients from healthy, uninfected individuals based on SARS-CoV-2 N protein serum levels, demonstrating its usefulness as a diagnostic test for COVID-19.

The present studies demonstrated the efficacy of a microfluidic immunosensor for rapid, high sensitivity measurements of SARS-CoV-2 N protein in serum. This assay utilizes a unique sensing scheme employing DMBs for immunomagnetic enrichment and signal amplification based on a simple magnetic enrichment process. The analytical performance of this assay was evaluated by performing measurements of human serum samples spiked with SARSCoV-2 N protein, which could be detected at concentrations as low as 10 pg/mL in 5× diluted serum within 30 min and 50 pg/mL in whole serum within 55 min. This immunosensor was also adapted for a smartphone-based diagnostic device, which does not require external pumps or power sources. Using this handheld device, SARS-CoV-2 N protein could be detected in 5× diluted serum and whole serum samples at concentrations as low as 100 and 230 pg/mL, respectively. The utility of this immunosensor was also assessed to detect COVID-19 infection by testing clinical serum specimens, which revealed that it can accurately distinguish PCR-positive COVID-19 patients from healthy, uninfected individuals based on SARS-CoV-2 N protein serum levels. The portability, simplicity, and high sensitivity of this immunosensor makes it a promising tool for point-of-care COVID-19 testing.

### Example 5—Design of an ACEF-Enhanced Electrochemical Magneto-Immunosensor

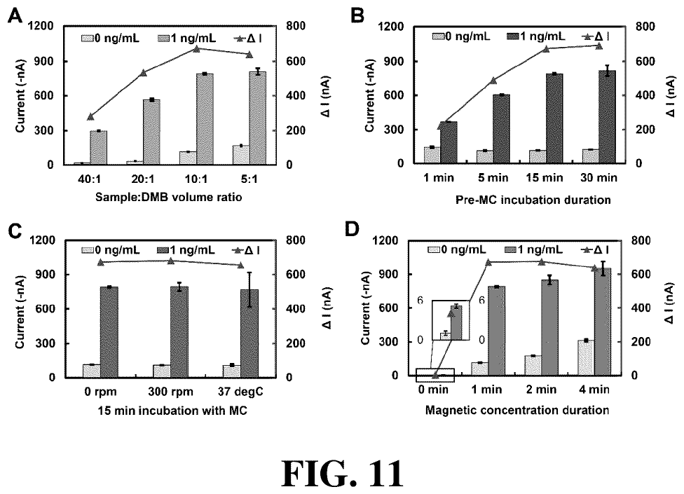

Many surface binding assays rely on diffusion-based mass transport to bring the relevant biomolecules (e.g., target analyte, detection antibody, reporter molecule) close to the reactive surface. For microwell immunoassays, such as ELISA, the distance that biomolecules need to travel to move from the bulk solution to the capture antibody-immobilized surface is several orders of magnitude larger than their diffusion length, necessitating long (˜1 h) incubation periods for mass transport.[35] Methods to enhance mass transport in microwell immunoassays, such as performing incubation at elevated temperatures and/or incorporating agitation, have been shown to offer moderate improvements in the analytical sensitivity and reductions in the assay time.[36] However, incorporating these methods with this magneto-immunosensor resulted in a negligible improvement in the sensor performance (FIG. 11C). Therefore, an alternative technique was employed to accelerate mass transport and enhance immunocomplex formation through the generation of electrothermally driven flows in the sample.

A schematic illustrating the design and working principle of immunosensor 120 is shown in FIG. 7. In this embodiment, immunosensor 120 is an ACEF-enhanced magneto-immunosensor. In the embodiment shown immunosensor 120 is a screen-printed gold electrode (SPGE) sensor comprising of an Au working electrode (WE) 121, Au counter electrode (CE) 122 and Ag/AgCl reference electrode (RE) 123. The WE is coated with anti-PfHRP2 IgM, which is used as the capture antibody. To initiate the measurement, the blood sample is mixed with DMBs and dispensed onto the sensor. DMBs are coated with horseradish peroxidase (HRP) and HRP-conjugated anti-PfHRP2 IgG, which is used as the detection antibody. If the target antigen is present in the sample, it binds to the DMB, forming an antigen-DMB immunocomplex. An AC potential is applied between the WE and CE for 5 min for ACEF mixing, which enhances mass transport and promotes the formation of the antigen-DMB immunocomplexes (FIG. 7A). After 4 min of ACEF mixing, a magnet is placed under the sensor, which generates a localized magnetic field, causing the antigen-DMB immunocomplexes to rapidly migrate to the sensor surface where they subsequently bind to the capture antibody-immobilized WE (FIG. 7B). In the presence of TMB substrate, HRP immobilized on the DMB catalyzes the reduction of H2O2 coupled to TMB oxidation. The oxidized TMB is reduced upon the application of a bias potential, generating an amperometric current that is proportional to the concentration of target antigen attached to the sensor surface and is detected by a detector 170 as shown in FIG. 7C. In the embodiment shown in FIG. 7C, detector 170 is an electrochemical analyzer configured to detect an amperometric current. As discussed further below, in other embodiments detector 170 can be configured as an optical detector configured to detect a colorimetric signal. Since each DMB contains multiple HRP molecules, an amplified amperometric signal is generated during the electrochemical reaction, enabling the detection of very low protein concentrations. If the sample does not contain the target antigen, then the DMBs are washed away from the sensor surface and a negligible electrochemical signal is generated upon the application of a bias potential in the presence of TMB substrate. The entire detection process is completed in 7 min.

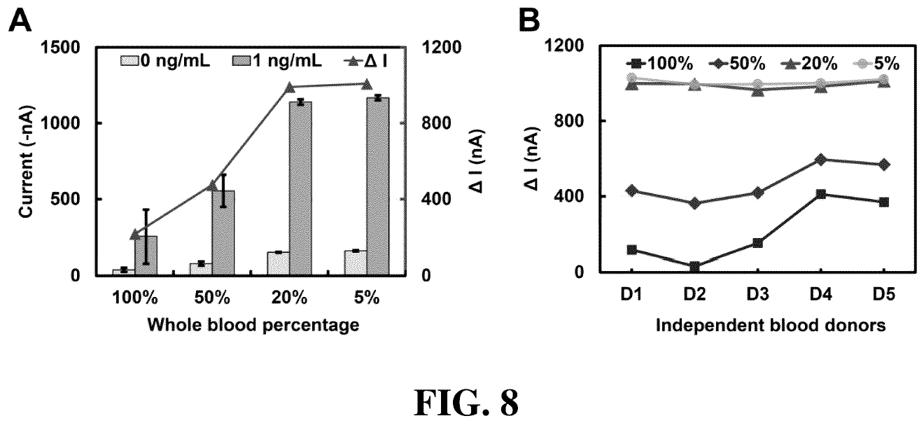

Influence of Blood Dilution on Immunosensor Performance: The use of whole blood for high sensitivity protein detection is challenging due to sample matrix effects. Whole blood is one of the most complex biological matrices since it contains a multitude of cellular and biomolecular components, which can cause interference in immunoassays and diminish the analytical performance.[37] The high viscosity of whole blood can also alter the protein binding efficiency[38] and variations in blood viscosity and ionic composition (pH) among different individuals[39,40] can lead to inconsistent results. Therefore, immunoassays generally involve sample preparation procedures to remove interfering components from blood to reduce matrix effects. Centrifugation is frequently used to separate serum or plasma from whole blood to reduce sample matrix effects and enhance the assay sensitivity. However, centrifugation is labor intensive and requires the use of bulky machinery. To circumvent the need for centrifugation, it was investigated whether blood matrix effects could be reduced by simply diluting the sample. Measurements of whole blood with varying dilution factors (0×, 2×, 5× and 20×) spiked with PfHRP2 at 0 ng/mL and 1 ng/mL were performed to investigate the effect of blood dilution on the performance of the immunosensor. As shown in FIG. 8A, samples with higher dilution factors generated larger values of ΔI, which represents the difference in the amperometric signal between the positive (1 ng/mL) and negative (0 ng/mL) controls. Specifically, the 2× and 5× diluted blood samples generated ˜2-fold and ˜5-fold larger ΔI values, respectively, than those generated from the undiluted blood sample, indicating that sample dilution can significantly diminish blood matrix effects. Diluting whole blood beyond 5× did not result in a noticeable improvement in sensor performance. These results demonstrate that a 5× dilution factor effectively reduces blood matrix effects for this immunosensor.

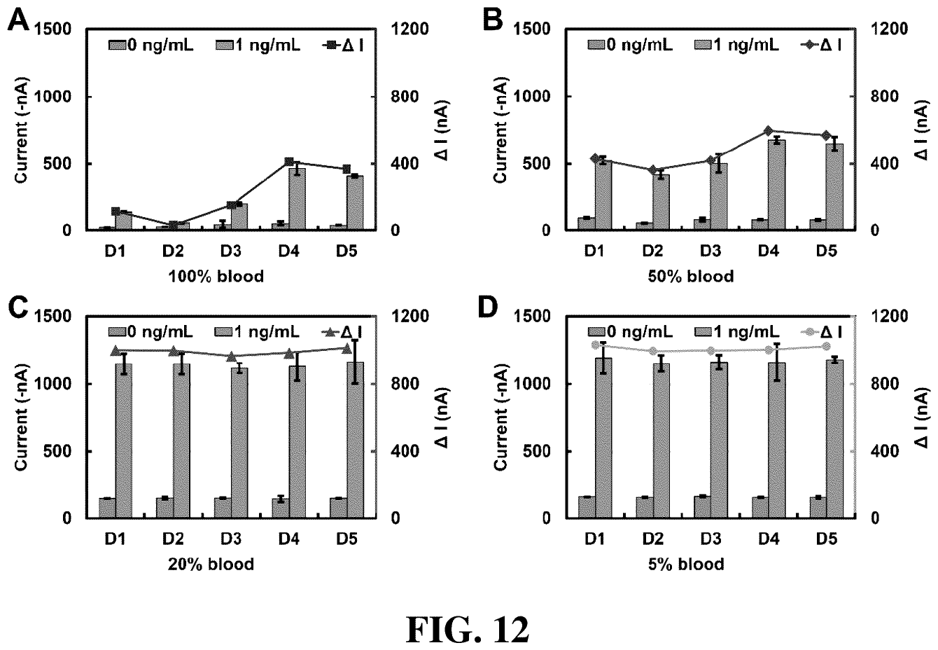

The influence of blood dilution was also studied on the reliability of the immunosensor by performing measurements of spiked blood samples, with varying dilution factors, obtained from five independent donors. ΔI values generated from the donor samples with different dilution factors are plotted in FIG. 8B (amperometric signals generated from the positive and negative controls from which the ΔI values were determined are presented in FIG. 12). The undiluted and 2× diluted blood samples exhibited very large variations in ΔI values, which were attributed to the differences in blood (e.g., viscosity, ionic composition) among the different donors. These variations can affect both the ACEF mixing efficiency and protein binding kinetics, which can subsequently alter the response of the sensor. In contrast, the 5× and 20× diluted blood samples generated consistent ΔI values for all five donor samples with a coefficient of variation of <2%. While 20× diluted blood generated ΔI values that were marginally more consistent that those generated by 5× diluted blood, excessive sample dilution can lower the concentration of the target analyte below the LOD of the sensor, effectively diminishing the sensitivity of the assay. Therefore, a 5× dilution factor ensures that this immunosensor generates consistent results when testing blood samples from different individuals while maintaining a high analytical sensitivity.

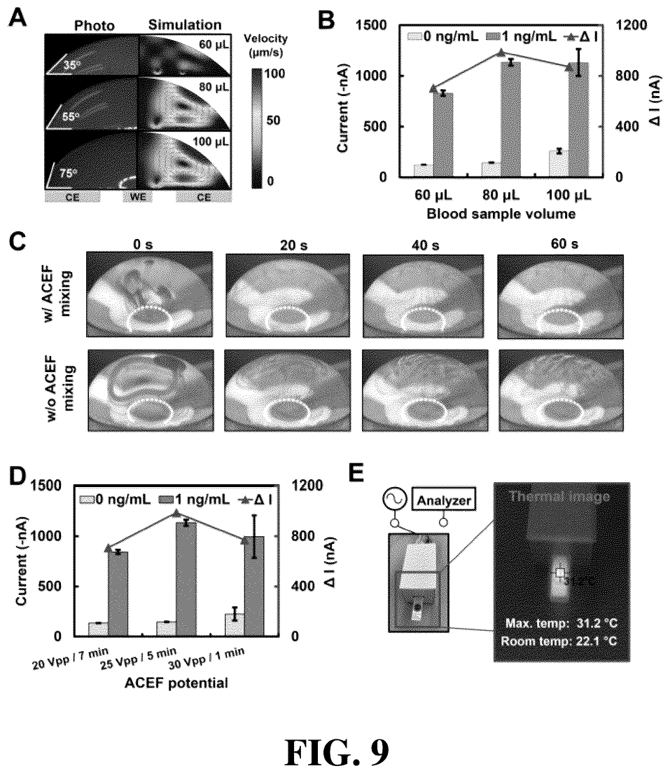

AC Electrothermal Flow Characterization and Optimization. Numerical simulations were performed to study the characteristics of electrothermally induced flow using a three-electrode configuration and investigate the influence of the sample volume on the electrothermal flow properties. As shown in FIG. 9A, the blood sample forms a droplet on the sensor surface and the shape of the droplet is guided by the sample volume. When an AC potential is applied to the sensor, swirling microflows are generated within the droplet between the WE and CE. The simulation results show that the electrothermal flow velocity is influenced by the sample volume, where larger droplets exhibit faster flow velocities. Experimental studies were carried out to measure the amperometric signals generated from blood samples, with varying volumes, spiked with PfHRP2 at 0 ng/mL and 1 ng/mL. As shown in FIG. 9B, the ΔI values generated from the 80 μL droplet were ˜40% larger compared with those generated from the 60 μL droplet, demonstrating that faster electrothermal flow can lead to improved sensor performance However, the ΔI values generated from the 100 μL droplet were ˜12% lower than those generated from the 80 μL droplet. It was hypothesized that with excessively fast flow velocities, the surface-immobilized blocking proteins become detached from the sensor surface, leading to an increase in nonspecific binding, as indicated by the ˜2× higher background signals that were generated for the 100 μL droplet compared with the 80 μL and 60 μL droplets.

To visualize electrothermally induced fluid motion, red microbeads were used as tracer particles and added to a 1×PBS droplet on a SPGE sensor that was stimulated by an AC signal. As shown in FIG. 9C, the beads are immediately pulled into the swirling flows within 5 s of being dispensed onto the droplet. Within 20 s, the beads move throughout the entire droplet following the streamlines of the flow. The motion of the beads is consistent with the velocity fields predicted by the numerical simulations (FIG. 9A). This is the first time that ACEF motion has been experimentally visualized and reported in literature. The rapid swirling motion generated by AC electrothermal flow leads to vigorous mixing, which enhances mass transport within the droplet and promotes antigen-antibody reactions. Without ACEF mixing, the motion of the beads is largely directed by buoyancy and diffusion, which causes them to disperse on the surface of the droplet shortly after being dispensed onto the droplet. After ˜20 s, the beads exhibit minimal movement within the droplet (FIG. 9C). These results also revealed that particles several microns in diameter can be transported across relatively large distances using AC electrothermal flow, validating its effectiveness for transporting smaller particles, such as proteins and magnetic beads (DMBs). While the electrothermally driven flows generated in this work were limited to buffer and blood samples, electrothermal flows can also be generated in other biological fluids, such as saliva and urine, further expanding the utility of this method for the development of other types of rapid diagnostic tests.

The ACEF mixing parameters were optimized by performing measurements of blood spiked with PfHRP2 at 0 ng/mL and 1 ng/mL using varying potentials (20 Vpp, 25 Vpp and Vpp) and durations (1 min, 3 min, 5 min, 7 min, 9 min and 11 min). Prior studies have shown that AC frequencies>100 kHz are necessary for generating electrothermally induced flow[22,41] and that frequencies between 200 kHz and 15 MHz result in similar ACEF performance.[21,42] Therefore, 200 kHz was selected for this work.

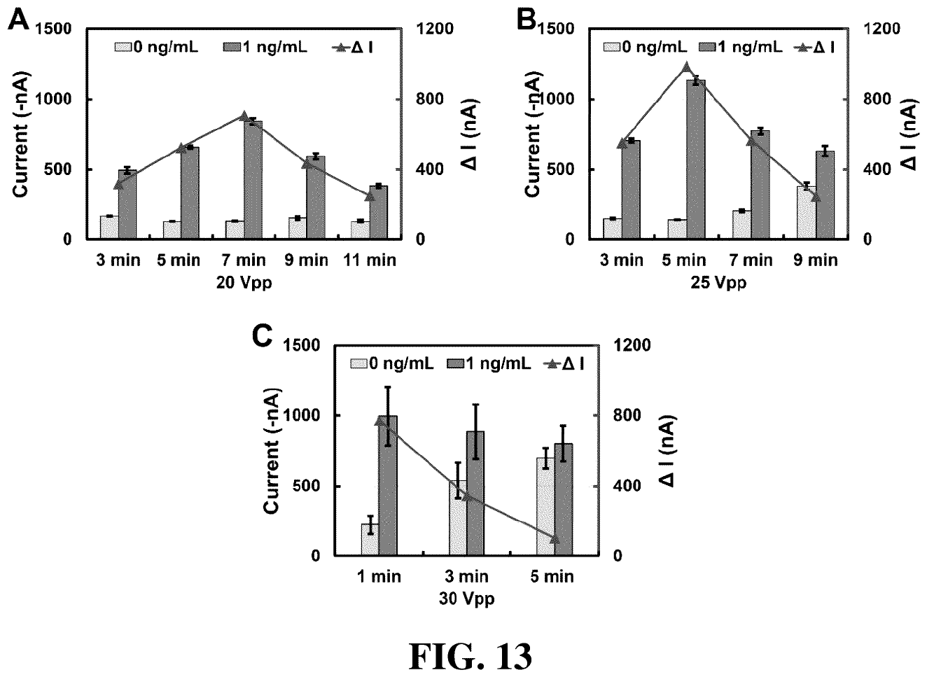

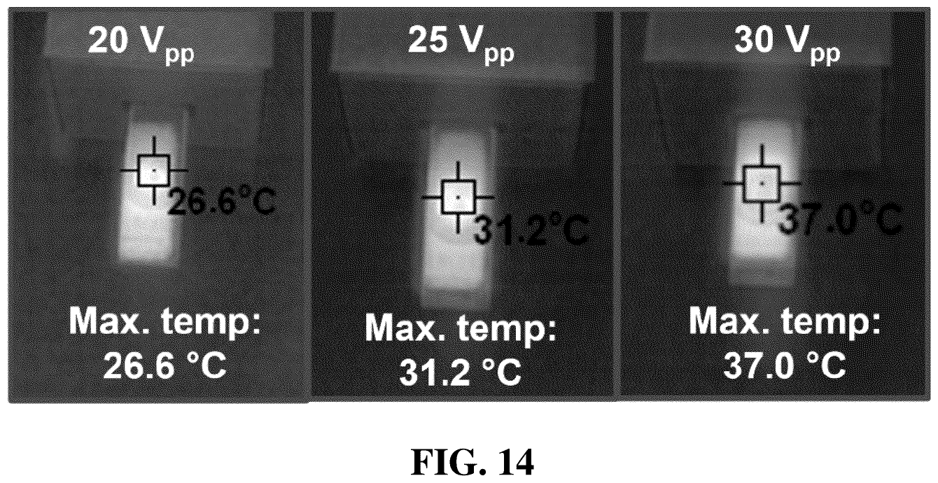

Amperometric signals and ΔI values for all of the tested parameters are presented in FIG. 13 and the data for the highest performing parameters are plotted in FIG. 9D. The largest ΔI values were generated by applying 25 Vpp for 5 min, which were ˜40% larger than those generated by applying 20 Vpp for 7 min. These results demonstrate that higher AC potentials can lead to an improvement in the sensor performance, even with a shorter duration. However, there was a drop in ΔI (and rise in the background signal) when using 30 Vpp for 1 min. Since higher AC potentials generate faster electrothermal flows in the droplet, this can cause the surface-immobilized blocking proteins to become detached from the sensor surface, leading to an increase in nonspecific binding. The amount of Joule heating produced during ACEF mixing was also studied by measuring the temperature of blood droplets using a thermal imaging camera. As shown in FIG. 14, the droplet temperature is proportional to the AC potential where larger potentials resulted in higher droplet temperatures. Using the optimized ACEF mixing parameters (25 Vpp, 200 kHz, 5 min), a maximum droplet temperature of 31.2° C. (FIG. 9E) was measured, which is within normal physiological conditions and should not negatively affect the integrity or binding kinetics of proteins in the blood sample.

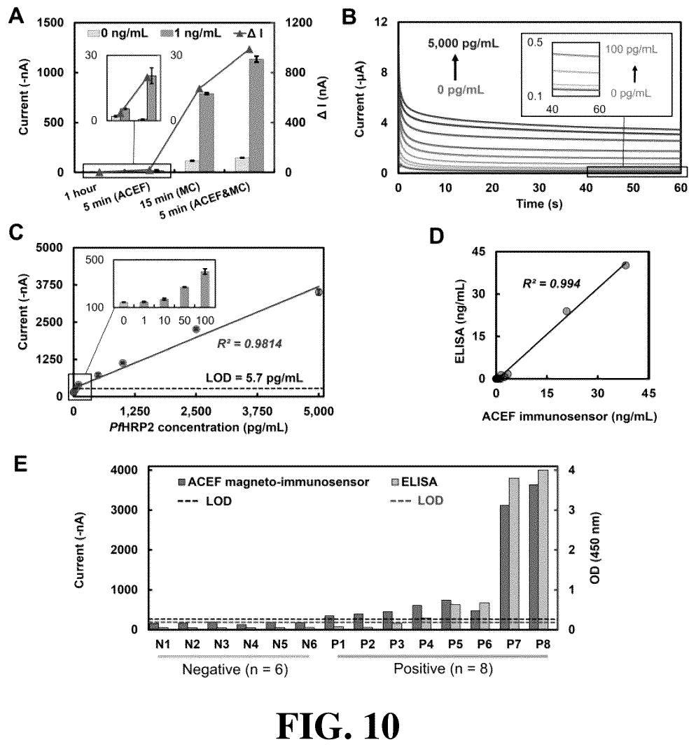

Performance of the ACEF-Enhanced Magneto-Immunosensor. The improvement in the sensor performance was first evaluated by incorporating ACEF mixing with the electrochemical magneto-immunosensor. Measurements of blood spiked with PfHRP2 at 0 ng/mL and 1 ng/mL were performed using the magneto-immunosensor with or without ACEF mixing. The assay parameters for the magneto-immunosensor were optimized. Measurements were also performed with ACEF mixing only (without magnetic concentration) and with 1 h of sample incubation (without ACEF mixing or magnetic concentration). The amperometric signals and ΔI values generated with the different sensor enhancement methods is presented in FIG. 10A. Measurements performed with 5 min of ACEF mixing (without magnetic concentration) resulted in a ˜7-fold increase in the ΔI values compared with those generated with 1 h of incubation; however, the magnitude of the amperometric signals generated by both methods was extremely low (10's of nA). A significant improvement in the sensor performance was attained using magnetic concentration only, which generated ΔI values that were ˜30-fold larger than those generated with ACEF mixing only. Combining ACEF mixing with magnetic concentration resulted in the largest ΔI values, which were ˜50-fold larger than those generated with ACEF mixing only and 1.5-fold larger than those generated with magnetic concentration only.

The analytical sensitivity (lower LOD) of the ACEF-enhanced magneto-immunosensor was assessed by performing measurements of blood spiked with increasing concentrations of PfHRP2. Chronoamperograms generated from the blood samples are presented in FIG. 10B, which shows a positive correlation between the amperometric current and the PfHRP2 concentration. The calibration curve is presented in FIG. 10C, which shows that this immunosensor exhibited a linear response from 0 to 5,000 pg/mL with a R² correlation coefficient of 0.9814. The calculated limit of detection of this immunosensor was 5.7 pg/mL, which is several orders of magnitude lower than that of commercially available ELISA tests using whole blood samples.^{[43-45]} In addition, each measurement was completed in 7 min, which is at least 20× faster than conventional ELISA and 7-30× faster than previously reported immunoassays capable of high sensitivity protein detection in whole blood.^{[12-14]}

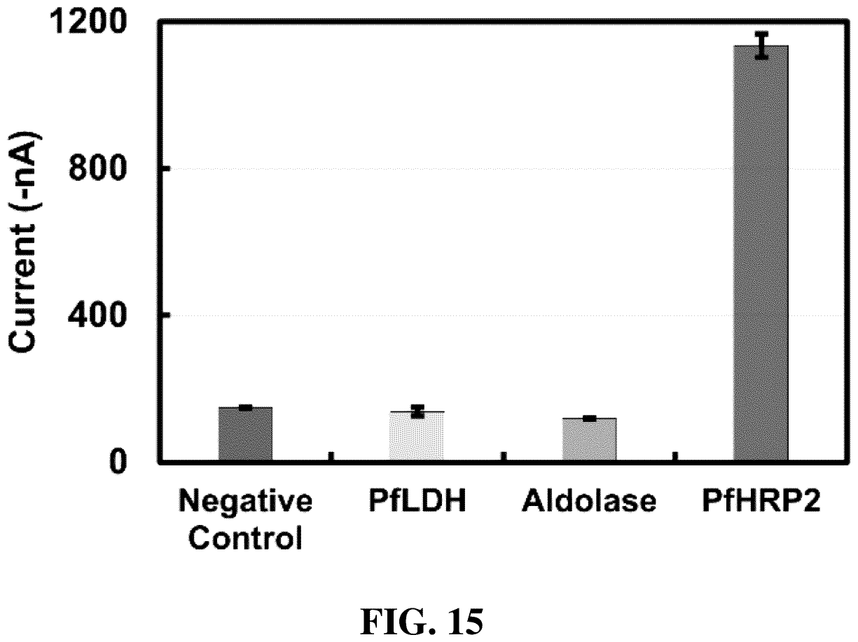

The selectivity of this immune sensor was evaluated by performing measurements of blood spiked with PfHRP2, pan-Plasmodium aldolase or P. falciparum lactate dehydrogenase (PfLDH) and non-spiked blood. As shown in FIG. 15, the amperometric signals generated from the samples containing PfLDH and aldolase were similar to those generated from the non-spiked blood sample, which was used as a negative control. In contrast, the amperometric signals from the sample containing PfHRP2 was ˜8-fold larger, indicating that this immunosensor is highly selectivity and will not cross-react with other biomarkers associated with P. falciparum infection.

PfHRP2 Quantification in Clinical Blood Samples. To evaluate the accuracy of this immunosensor, eight clinical blood samples obtained from malaria patients in Uganda confirmed by microcopy (P1-P8) and six blood samples obtained from healthy, uninfected donors from the U.S. (N1-N6) were analyzed. PfHRP2 measurements were performed on paired blood samples using the immune sensor and a commercial Cellabs Quantimal™ ultra-sensitive PfHRP2 ELISA kit. The PfHRP2 concentration determined by both methods are plotted in a scatter plot (FIG. 10D) and linear regression analysis showed that measurements generated by this immunosensor are highly correlated (R²=0.994) with those generated by the commercial ELISA kit over a large range of PfHRP2 levels from 0 to 40 ng/mL. Next, the utility of this immune sensor for diagnosing individuals with P. falciparum infection based on PfHRP2 measurements in whole blood was evaluated Amperometric signals generated by our immunosensor are plotted against the absorbance values generated by the Cellabs ELISA kit for all 14 clinical samples (FIG. 10E). A cut-off value of −280 nA was used for discriminating between malaria-positive and malaria-negative cases, which is the amperometric current at the calculated lower LOD of the immunosensor. As shown in FIG. 10E, the amperometric signals and absorbance values generated from all six uninfected donor samples (N1-N6) were below the cut-off values for both assays, indicating that both methods were able to accurately identify all the negative cases. When analyzing the malaria-positive samples (P1-P8), the ELISA kit was only able to identify five of the eight samples as positive cases based on the cut-off value specified by the manufacturer. In contrast, the amperometric signals generated from all eight positive samples were above the cut-off value of the immunosensor, indicating that was able to identify positive cases with better accuracy than the commercial ELISA kit.

In summary, an ultra-fast biosensor is provided herein that combines ACEF mixing with an electrochemical magneto-immunoassay for high sensitivity detection of protein biomarkers in whole blood. Through numerical simulation and measurements of PfHRP2 in whole blood, it was show that ACEF mixing resulted in enhanced transport of proteins and DMBs in the sample, which facilitates antigen-antibody interactions and promotes the formation of antigen-DMB immunocomplexes. The synergetic effects of ACEF mixing and immunomagnetic enrichment leads to a larger number of antigen-DMB immunocomplexes attached to the sensor surface within a very short amount of time, giving rise to enhanced amperometric signal generation. Furthermore, by circumventing the need for sample purification and multiple washing and incubation steps, this immune sensor offers improved ease of use compared to conventional immunoassays, making it particularly useful for rapid testing or point-of-care testing. This device can be readily adapted to detect other clinically relevant biomarkers by replacing the capture and detection antibody with different bioreceptors, thereby expanding its utility for rapid disease diagnosis and screening.

### Example 6—Materials and Chemicals

Dimethyl sulfoxide (DMSO), phosphate-buffered saline (PBS, pH 7.4), (ethylenedinitrilo) tetraacetic acid (EDTA), 2-Iminothiolane hydrochloride, horseradish peroxidase (HRP), and 3,3′,5,5′-Tetramethylbenzidine(TMB)substrate (T4444) were purchased from Sigma-Aldrich (St. Louis, MO). N-(3-Dimethylaminopropyl)-N′-ethylcarbodiimide (EDC), N-Hydroxysuccinimide (NHS) were obtained from Thermo Fisher Scientific (Waltham, MA). Stabil Block immunoassay stabilizer, StabilCoat Plus immunoassay stabilizer, and StabilZyme HRP stabilizer were purchased from SurModics, Inc. (Eden Prairie, MN). Carboxylated magnetic nanobeads (200 nm) were purchased from Ademtech (Pessac, France). Reagent diluent (10×, 10% bovine serum albumin (BSA) in 10×PBS) was purchased from R&D Systems (MN, USA). Mouse monoclonal anti-PfHRP2 IgM and anti-PfHRP2 IgG were purchased from ICL, Inc. (Portland, OR). Recombinant P. falciparum histidine-rich protein 2 (PfHRP2), P. falciparum lactate dehydrogenase (PfLDH), and pan-Plasmodium aldolase antigen were purchased from CTK Biotech (San Diego, CA). Human blood samples from healthy donors obtained in the U.S. were purchased from BioIVT (NY, USA). Blood samples from donors with P. falciparum infection obtained in Uganda under IRB/EC approval for general research use were purchased from Discovery Life Sciences (Huntsville, AL). All human samples were de-identified of all identifying information.

Preparation of Dually-Labeled Magnetic Nanobeads. 1 mg of carboxylated magnetic nanobeads was dispensed in 400 μL of MES buffer (pH 5.0, 25 mM) and washed twice. Next, 100 μL of MES buffer containing HRP and anti-PfHRP2 IgG at a 200:1 molar ratio was mixed with the nanobeads preactivated with 10 mg/mL of EDC/NHS and incubated overnight at room temperature. After washing with PBS and blocking of nonspecific binding sites with StabilCoat Plus stabilizer, the DMBs were dispersed in 400 μL of StabilZyme HRP stabilizer to a final concentration of 2.5 mg/mL and used immediately or stored at 4° C. for future use.

Preparation of Immunosensors. Laser-cut 100-μm-thick polyethylene terephthalate (PET) (McMaster-Carr) film with a 4 mm diameter opening was bonded to screen-printed gold electrode (SPGE) sensors (Metrohm Dropsens, Asturias, Spain) using double-sided adhesive tape (Adhesives Research, PA). Anti-PfHRP2 IgM was first thiolated by incubating 100 μL of antibody at 100 μg/mL with 100-fold molar excess of 2-iminothiolane in PBS containing 2 mM of EDTA for 1 h at room temperature, followed by centrifugal filtration (10 kDa, Amicon Ultra mL) for 5 and 10 min at 13,800 g to remove excess reagent. Thiolated anti-PfHRP2 IgM was immobilized on the SPGE sensor by incubating 2 μL of antibody solution at 200 μg/mL on the working electrode (WE) for 2.5 h at room temperature, followed by rinsing with PBS and gently drying with purified N2. StabilBlock stabilizer solution was dispensed on the sensor and dried at room temperature to passivate the surface and enhance the stability of the immobilized antibody. Sensors were used immediately or stored in sealed pouches with desiccants at 4° C. for future use.

ACEF Mixing and Electrochemical Measurements. 8 μL of DMB solution was mixed with 80 μL of whole blood spiked with PfHRP2 in a microcentrifuge tube, vortexed for 5 s, and 80 μL of mixed sample was dispensed on the sensor. Spiked blood samples were either used as is or diluted in 1× reagent diluent. ACEF mixing was performed by applying a 25 Vpp (peak-to-peak) potential at 200 kHz between the WE and CE for 5 min using a function generator (33522B, Keysight) and voltage amplifier (HVA200, Thorlabs). At the 4th min of ACEF mixing, the SPGE sensor was placed on a 4 mm neodymium magnet (McMaster-Carr) for 1 min The sensor was rinsed in lx PBS for 10 s and gently dried with N2, followed by application of 50 μL of TMB substrate on the sensor. After 1 min, chronoamperometric measurements were performed using a PalmSens4 potentiostat by applying a bias potential of −0.2 V (vs. Ag/AgCl) for 60 s. Current values were obtained at 60 s of chronoamperograms.

PfHRP2 Detection in Clinical Blood Samples. PfHRP2 measurements were performed using a Quantimal™ ultra-sensitive PfHRP2 ELISA kit (Cellabs, Australia). Blood samples were diluted 5-fold in 1× reagent diluent. Measurements were performed according to the manufacturer's instructions and absorbance values were measured at OD 450 using a BioTek Epoch microplate spectrophotometer. The cut-off value for discriminating positive from negative cases was determined as the absorbance value of negative control plus 0.1 OD according to the manufacturer's protocol. PfHRP2 measurements were performed using the ACEF-enhanced magneto-immunosensor as described above using 5× diluted blood sample.

Numerical Simulation of AC Electrothermal Flow. AC electrothermal flow was simulated using COMSOL Multiphysics software by coupling AC electric field and heat transfer to obtain the 2-dimentional (2D) axisymmetric velocity profile in a liquid droplet. The electrothermal force induced by gradients of permittivity ε and conductivity σ (=1.6 S/m, 1×PBS) can be written as:[23]

$${\vec{F}}_{E} = - 0.5 [ ( \frac{∇ σ}{σ} - \frac{∇ ε}{ε} ) \vec{E} \frac{ε \vec{E}}{1 + {( ω τ )}^{2}} + 0.5 {\vert \vec{E} \vert}^{2} ∇ ε ] \qquad ( 1 )$$

where τ=ε/σ is the charge relaxation time of the fluid, EE [symbol] is the electric field, and ω is the frequency of the AC electric field. In this model, the buoyancy force generated by the density gradient was considered and is denoted by:

\vec{F}_{B}=ρ_{E}g (2)

where ρE is the instantaneous density of the fluid. Permittivity and density are simplified to be a function of temperature in the simulation.[24]

AC Electrothermal Flow Visualization. 6.0 μm red polystyrene microbeads (15714, Polysciences) were used as tracer particles to visualize the flow patterns within a liquid droplet with and without ACEF mixing. 80 μL of 1% BSA in 1×PBS was dispensed onto the sensor followed by the application of 25 Vpp at 200 kHz between the WE and CE. 2 μL of the stock microbead solution was dispensed onto the droplet and the motion of the microbeads was recorded using a digital microscope (VHX-7000, Keyence).

Optimization of the Magneto-Immunosensor. Experiments were performed to optimize several assay parameters, including the sample to DMB solution ratio, the pre-magnetic concentration incubation duration and incubation condition, and the magnetic concentration duration, for the electrochemical magneto-immunosensor (without ACEF mixing) for PfHRP2 detection in whole blood. The sample to DMB solution ratio was optimized by performing measurements of 5× diluted whole blood spiked with PfHRP2 at 0 ng/mL or 1 ng/mL using different sample to DMB volume ratios ranging from 5:1 to 40:1. The amperometric signals and ΔI values generated for each volume ratio are plotted in FIG. 11A, which shows that the magnitude of the amperometric signals increases steadily with decreasing sample to DMB volume ratios from 40:1 to 10:1. However, measurements using a sample to DMB ratio of 5:1 resulted in a significantly higher background signal relative to the detection signal, causing the ΔI value to decline compared with that obtained using a 10:1 sample to DMB ratio. This is likely due to the presence of an excessive amount of DMBs in the sample-DMB mixture, which leads to more nonspecific binding of the DMBs on the sensor surface. Therefore, a sample to DMB volume ratio of 10:1 was selected as the optimal condition. The pre-magnetic concentration incubation duration was optimized by performing measurements of 5× diluted whole blood spiked with PfHRP2 at 0 ng/mL or 1 ng/mL using varying incubation durations. The amperometric signals and ΔI values generated for each incubation duration are plotted in FIG. 11B. This data shows that longer incubation times generate larger ΔI values until steady state is reached at 15 min, which was selected as the optimal incubation duration. The influence of the pre-magnetic concentration incubation condition on the sensor performance was studied by performing measurements of 5× diluted whole blood whole blood spiked with PfHRP2 at 0 ng/mL or 1 ng/mL using three different incubation conditions: 1) room temperature with orbital shaking at 300 rpm, 2) room temperature without agitation, and 3) 37° C. without agitation. As shown in FIG. 11C, the amperometric signals and ΔI values generated for all three incubation conditions are similar, which indicates that the use of agitation or elevated temperatures has a negligible effect on the performance of the magneto-immunosensor.

The last parameter that was optimized was the magnetic concentration duration, which was carried out by performing measurements of 5× diluted whole blood spiked with PfHRP2 at 0 ng/mL or 1 ng/mL with varying durations of magnetic concentration. Without magnetic concentration, the generated amperometric signals and ΔI values are similar to those of the background signal. Applying magnetic concentration for 1 min resulted in a considerable increase in the ΔI values by ˜170-fold, compared with those generated without magnetic concentration. Applying magnetic concentration for durations >1 min resulted in a minimal increase in the amperometric signals; however, the background signals further increased relative to the detection signals, causing the ΔI values to decrease. Therefore, 1 min was selected as the optimal magnetic concentration duration.

### Example 7—Rapid magneto-enzyme-linked immunosorbent assay for ultrasensitive protein detection

The detection and quantification of protein biomarkers is used for a broad range of clinical applications, including disease diagnosis and screening, assessing therapeutic response, and monitoring disease progression [46-49]. The current gold standard technique for quantitative protein detection in clinical specimens is enzyme-linked immunosorbent assay (ELISA). ELISA offers the benefits of high sensitivity measurements, with most commercial ELISA kits claiming a lower limit of detection (LOD) in the 100's of pg mL⁻¹ range [50], and high specificity, resulting from its use of antigen-antibody pairs. In addition, ELISA can process many samples at once due to its format in a 96-well plate, making it useful for large-scale testing or blood screening. Due to these advantages, ELISA is recommended by the World Health Organization as an essential diagnostic modality [51], and as such is widely available in many diagnostic laboratories worldwide. While ELISA offers many benefits as a diagnostic technique, one of its main drawbacks is that it involves multiple incubation and wash steps, making the overall procedure laborious and time-consuming (˜3-4 hours per test). The extended time and person-hours required for conventional ELISA hinder its use for applications requiring short turnaround times, such as on-site diagnostic testing or high-throughput screening.

To reduce the time and complexity associated with ELISA, various techniques have been developed to enhance the kinetics of antigen-antibody binding, amplify the detection signal produced by the enzymatic reporter or simplify the testing protocol. Dixit, et al. employed covalent immobilization of the capture antibody in the microwell which, when compared to passive adsorption, resulted in a ˜10× improvement in the LOD for the detection of human fetuin A [52]. Additionally, nanoparticles have been used as carriers for detection antibodies and/or reporter molecules, which can amplify the detection signal due to their large surface area and presence of multiple active binding sites, allowing them to carry a large

number of detection molecules [53]. Ambrosi et al. demonstrated that the use of gold nanoparticles coated with dAb-reporter conjugates resulted in 2-fold higher sensitivity for detecting Cancer Antigen 15-3 with a significantly reduced enzymatic reaction time compared with the use of free detection antibody (dAb) [54]. Magnetic nanoparticles offer the further advantage of localization, as they can be rapidly concentrated using an external magnetic field. This facilitates the transport of biomolecules in the sample, which reduces the time needed for immunocomplex formation and enables rapid, simple separation/concentration of biological species within an immunoassay. This technique has been demonstrated for the rapid transfer of magnetic bead conjugated immune complexes between reagents and wash buffers, enabling the detection of anti-SARS-CoV-2 antibodies within 15 minutes [55]. In addition to reducing the assay time, the use of magnetic nanoparticles has also been shown to enhance the colorimetric signal of ELISA by transferring analyte-magnetic bead complexes from a large sample volume, thus concentrating a dilute analyte within a small volume at the microwell surface [56]. These techniques have leveraged the ability of an external magnetic field to rapidly isolate magnetic bead complexes, resulting in lower LODs and faster biomarker detection.

An alternative strategy to enhance the analytical sensitivity of ELISA has been to modify the enzyme reporter. In conventional ELISA protocols, horseradish peroxidase (HRP) is used as the enzyme reporter and undergoes an oxidation reaction in the presence of 3,3′,5,5′-Tetramethylbenzidine (TMB) substrate, resulting in a colorimetric signal that is proportional to the concentration of the target analyte on the microwell surface. Therefore, increasing the amount of HRP that is attached to the target analyte can amplify the detection signal, allowing for lower protein concentrations to be detected. This was demonstrated by Wang, et al. who functionalized nanoparticles with biotin and poly(amidoamine) to bind additional HRP molecules, which increased the detection signal by 10× [57]. Similarly, de la Sema et al. reported the use of poly-HRP, a polymeric unit of HRP that produces a color change equivalent to multiple molecules of HRP, in conjunction with magnetic nanobeads for the detection of Plasmodium falciparum lactate dehydrogenase (PfLDH) in lysed whole blood. This modified ELISA exhibited a LOD of 0.11 ng mL⁻¹ and assay time of 1 hour [58], indicating that increasing the concentration of enzymatic reporter can improve the sensitivity and reduce the time required for protein detection.

The approaches described above have been successful in either enhancing the analytical sensitivity of ELISA, reducing the assay time, or simplifying the testing protocol; however, there is currently no ELISA test that can offer ultrasensitive (single pg mL⁻¹) protein quantification in clinical samples in ≤30 min. While there are other types of rapid immunoassays (e.g., chemiluminescence, fluorescence, electrochemical [59, 60]) that can detect proteins with high sensitivity, they require specialized instrumentation, involve complicated protocols, or are not suitable for testing large numbers of samples at once. To overcome these limitations, a rapid (30 min) magneto-ELISA has been developed for ultrasensitive protein measurements in purified and whole blood samples which does not require specialized instrumentation and is compatible with standard microplate readers and ELISA protocols, as disclosed herein. This novel assay utilizes dually labeled magnetic nanoparticles (DMPs) that are coated with HRP and an HRP-conjugated dAb. Each DMP contains multiple dAb molecules, increasing the number of binding sites for the target antigen, as well as multiple HRP molecules, resulting in a more substantial enzymatic reaction and amplified detection signal. Additionally, this assay utilizes a rapid and simple immunomagnetic enrichment technique to transport antigen-DMP immunocomplexes to the capture antibody (cAb)-immobilized microwell surface, which enhances the kinetics of sandwich immunocomplex formation. As disclosed herein, this magneto-ELISA can be readily adapted to detect other protein biomarkers in different types of clinical samples, including human plasma, serum, and whole blood, while maintaining high analytical sensitivity, showcasing its versatility as a diagnostic technique.

Materials and Method—Biochemicals and reagents: 10 mM Tris hydrochloride buffer (pH=8.0) and 0.01 M phosphate buffered saline (pH=7.4) were purchased from Bioworld Inc. and Sigma-Aldrich, respectively. 0.1 M 2-(N-morpholino) ethanesulfonic acid (MES, pH=4.7) buffer was purchased from Thermo Fisher Scientific and diluted to 25 mM using deionized water. Washing buffer was prepared by diluting Tween-20 (Sigma-Aldrich) in PBS to produce a 0.05% (w/v) Tween-20 solution. N-(3-dimethylaminopropyl)-N-ethylcarbodiimide (EDC) was purchased from Thermo Fisher Scientific. N-hydroxysuccinimide (NHS) and horseradish peroxidase (HRP) were purchased from Sigma-Aldrich. Enhanced K-Blue TMB substrate was purchased from Neogen Inc. Matrix Guard Diluent, StabilBlock Immunoassay Stabilizer, StabilCoat Plus Immunoassay Stabilizer and StaiblZyme HRP Conjugate Stabilizer were purchased from Surmodics Inc. Human sera was purchased from Sigma-Aldrich and human plasma and whole blood were purchased from BioIVT. Blood samples from donors with P. falciparum infection obtained in Uganda under IRB/EC approval for general research use were purchased from Discovery Life Sciences. All human samples were de-identified of identifying information.

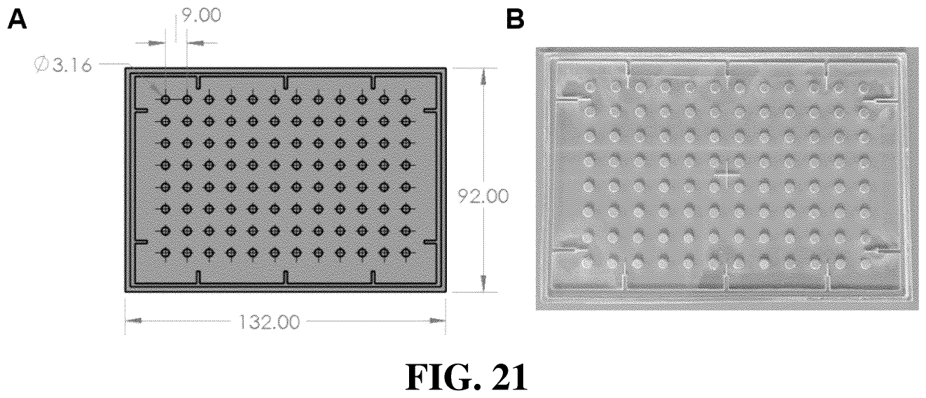

Fabrication of the magnetic stag: The magnetic stage consists of an array of 96⅛-inch diameter neodymium magnets (McMaster Carr) with centers positioned 9 mm apart in a laser-cut poly methyl methacrylate (PMMA) base that fits a standard 96-well plate (FIG. 21). The base consists of 3 layers of PMMA joined together using double-sided tape to provide a height of 3 mm, which ensures that each magnet is in contact with the bottom of the 96-well plate and centered under each well.

Preparation of microplate: Anti-Plasmodium falciparum histidine-rich protein 2 (PfHRP2) IgG (Fitzgerald Industries International) or anti-SARS-CoV-2 N protein IgG (Arigo Biolaboratories) was diluted in Tris-HC1 buffer to a concentration of 10 μg mL⁻¹. 45 μL of the diluted antibody solution was added to each well of a high-bind polystyrene 96-well plate (Corning), incubated at 4° C. for 16 hours to allow for passive adsorption of the antibody to the plate, then washed with 0.05% Tween-20. 300 μL of StabilBlock Immunoassay Stabilizer was added to each well, incubated for 1 hour, and removed by tapping the plate upside down. Prepared plates were dried overnight at 4° C. and used immediately or vacuum sealed and stored at 4° C. for up to one month.

Preparation of dually labeled magnetic particles (DMPs): DMPs were prepared by binding HRP and anti-PfHRP2 IgG-HRP conjugates (ICL Inc.) or anti-SARS-CoV-2 N protein IgG-HRP conjugates (GeneTex) to 200 nm carboxylated nanomagnetic beads (Ademtech) using carbodiimide chemistry, as previously described [61]. Briefly, 1 mg of magnetic beads was washed using 25 mM MES buffer and shaken at 500 rpm with 200 μL of EDC/NHS (10 mg mL⁻¹ in 25 mM MES) for 50 min. After washing, the beads were mixed with 50 μL of HRP-conjugated detection antibody (50 μg mL⁻¹) and 50 μL of HRP (3mg mL⁻¹) in 25 mM MES (1:200 IgG:HRP molar ratio). The bead-protein mixture was shaken overnight (15 hours), then washed six times with PBS, incubated twice with StabilCoat Plus Immunoassay Stabilizer for 45 min each, and stored in 400 μL of StabilZyme HRP Conjugate Stabilizer. DMPs were used immediately or stored at 4° C. for up to 2 weeks.

ELISA measurements: PfHRP2 (CTK Biotech), P. falciparum lactate dehydrogenase (PfLDH, CTK Biotech), Plasmodium aldolase (CTK Biotech) or SARS-CoV-2 N protein (Advaite, Inc) was spiked in human sera, plasma or whole blood diluted 10× in MatrixGuard diluent (Surmodics, Inc.) to generate simulated samples for assay optimization and testing. The simulated sample was first combined with DMPs at a 1:40 ratio. 85 μL of the sample-DMP mixture was added to each well and the plate was incubated on an orbital shaker for 14 min at 300 rpm. The plate was placed on the magnetic stage for 1 min for magnetic concentration and then incubated without agitation at room temperature for 5 min, followed by washing six times with 0.05% Tween-20. 100 μL of TMB substrate was added to each well and the plate was incubated on an orbital shaker for 10 min at 150 rpm. 50 μL of 2N H₂SO₄ was added to each well to stop the HRP-TMB reaction. The colorimetric signal was read using a BioTek Epoch microplate spectrophotometer at a wavelength of 450 nm. ELISA measurements of deidentified clinical blood samples from malaria-positive and malaria RDT-negative samples were performed using the same protocol as the simulated samples. Quantimal Ultrasensitive PfHRP2 ELISA kits were purchased from Cellabs Inc. and run according to the manufacturer's protocol. For comparison with the commercial kit, the concentration values obtained from the magneto-ELISA were scaled by a factor of 2.076. Concentrations below the detection range of the calibration curve were considered to have a concentration of 0 ng mL⁻¹ for both the magneto-ELISA and commercial kit.

Statistical analysis: Statistical analysis was conducted using an unpaired Student's t-test between different testing parameters and a Spearman's rank correlation coefficient for comparison to standard ELISA techniques. Data analysis was conducted using GraphPad Prism 9.

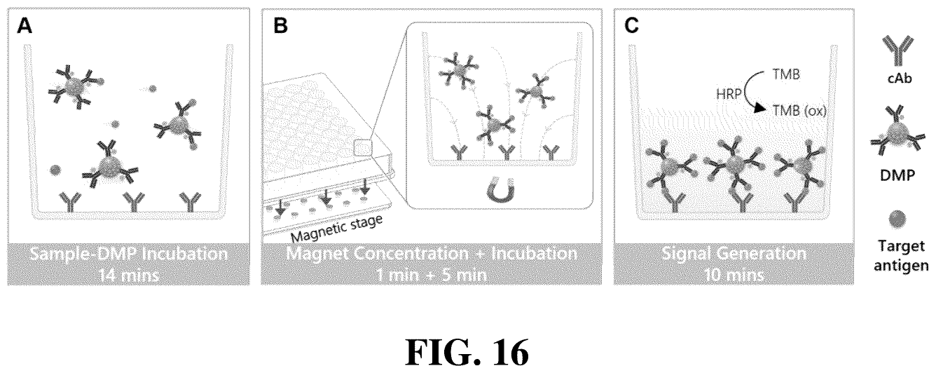

Results and Discussion—Principle of the magneto-ELISA: This assay is based on a conventional sandwich ELISA format where an antibody pair and enzyme reporter are used to detect a target antigen. Similar to a conventional sandwich ELISA, the cAb is immobilized on the bottom of the microplate well. However, our assay differs in its utilization of DMPs that are coated with HRP-conjugated dAb and free HRP, which allows for rapid immunomagnetic enrichment and enhanced signal amplification. To initiate the measurement, DMPs are added to the sample and the sample-DMP mixture is incubated in the wells (with agitation) for 14 min. If the sample contains the target antigen, it binds to the DMP and forms an antigen-DMP immunocomplex (FIG. 16 Panel A). The plate is then placed on the magnetic stage, which generates a localized magnetic field under each well and causes the antigen-DMP immunocomplexes to rapidly migrate to the bottom of the well where they subsequently bind to the surface-immobilized cAb (FIG. 16 Panel B). In the presence of TMB substrate, the HRP-coated DMPs catalyze the oxidation of TMB, generating a colorimetric signal that is proportional to the concentration of target antigen attached to the cAb-immobilized well (FIG. 16 Panel C). If the sample does not contain the target antigen, the DMPs are washed away from the well and a negligible colorimetric signal is generated upon application of the TMB substrate. With the exception of the magnetic stage, which has an estimated cost of approximately USD $9, this magneto-ELISA requires no additional parts or specialized instrumentation and is compatible with standard microplate readers.

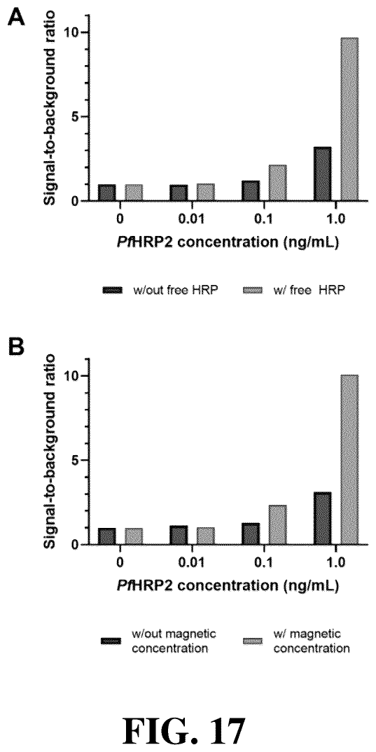

The use of DMPs in this magneto-ELISA offers two major advantages over conventional ELISA. First, enhanced signal amplification is achieved with reduced incubation times because the DMPs are coated with HRP-conjugated dAb and free HRP. Since the colorimetric signal is generated from the reaction between HRP and the TMB substrate, the large amount of HRP on each DMP enhances the enzymatic reaction for a single surface-immobilized immunocomplex, resulting in a more substantial colorimetric signal. The improvement in the colorimetric signal was evaluated by using magnetic particles coated with HRP-conjugated dAb and free HRP or magnetic particles coated with HRP-conjugated dAb only for measurements of PfHRP2 spiked in human sera. As shown in FIG. 17 Panel A, the signal-to-background ratios (SBRs) generated using magnetic particles coated with HRP-conjugated dAb and free HRP were up to 3-fold larger compared with those generated from the magnetic particles that contained HRP-conjugated dAb only. This result shows that immobilizing both free HRP and HRP-conjugated dAb on DMPs results in enhanced signal amplification without increasing the background signal, thereby improving the analytical sensitivity of the assay.

The other major advantage of this approach is that the immunomagnetic enrichment process accelerates the transport of antigen-DMP immunocomplexes to the bottom of the cAb-immobilized well, which enhances the immunoreaction kinetics, thereby increasing the likelihood of sandwich immunocomplex formation. The enhancement in immunocomplex formation due to magnetic concentration was studied by performing measurements of PfHRP2-spiked human sera using the magneto-ELISA with 1 min of magnetic concentration or with 30 min of incubation without magnetic concentration. As shown in FIG. 17 Panel B, SBRs generated with magnetic concentration were up to 3-fold larger compared with those generated without magnetic concentration with a negligible change in the background signal. Therefore, the use of magnetic concentration further enhances the detection signal of the assay, while significantly reducing the incubation time to 30 minutes (compared to ˜3-4 hours for conventional ELISA).

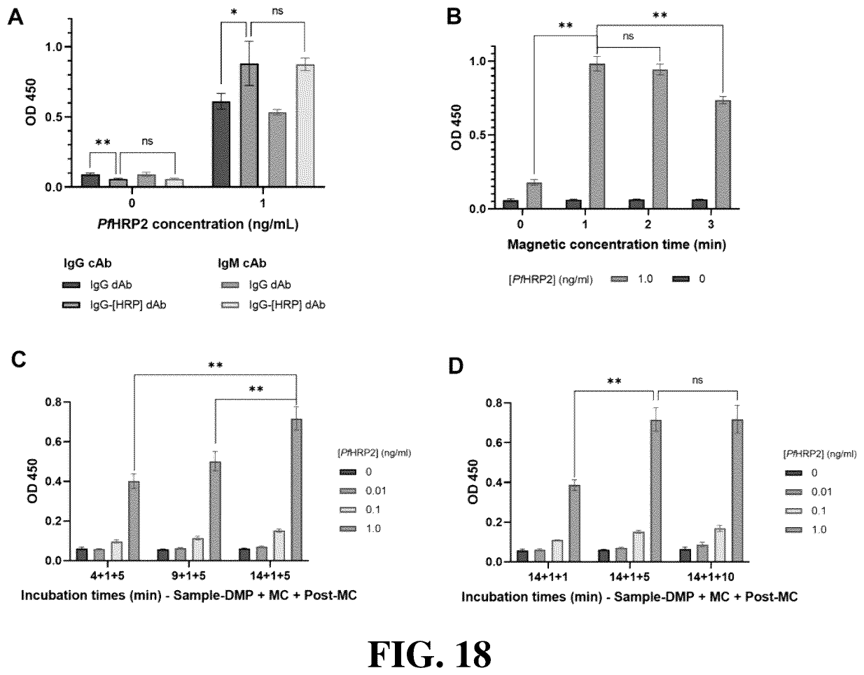

Optimization of assay parameters: Several assay parameters were optimized to maximize the SBR and minimize the variability in the detection signal. All assay optimization experiments were carried out using PfHRP2 as the target analyte. The affinity of PfHRP2 to the capture and detection antibodies was studied by performing measurements of human sera spiked with 1 ng mL⁻¹ or 0 ng mL⁻¹ of PfHRP2 using different anti-PfHRP2 antibody pairs. Both anti-PfHRP2 IgG and IgM produced similar absorbance values when used as the cAb. However, the use of an HRP-conjugated anti-PfHRP2 IgG dAb resulted in an ˜1.4-fold increase in the absorbance for the positive control sample (1 ng mL⁻¹) and a reduction in absorbance for the negative control sample (0 ng mL⁻¹) compared with those generated using anti-PfHRP2 IgG dAb (FIG. 18 Panel A). The higher absorbance values generated by the HRP-conjugated anti-PfHRP2 IgG are due to the additional HRP molecules bound to each DMP, thus increasing the enzymatic reaction and colorimetric signal produced by each surface-immobilized immunocomplex. Based on these results, anti-PfHRP2 IgG was selected as the cAb and HRP-conjugated anti-PfHRP2 IgG was selected as the dAb for the PfHRP2 assay.

The durations of sample-DMP incubation, magnetic concentration, and post-magnetic concentration incubation were optimized to maximize the analytical sensitivity while reducing the overall assay time. First, the magnetic concentration duration was studied by performing measurements of PfHRP2 spiked in human serum using varying magnetic concentration durations, which revealed that 1-2 min generated the highest absorbance values for all PfHRP2 concentrations (FIG. 18 Panel B). It was observed that magnetic concentration durations>2 min resulted in lower absorbance values since longer concentration times can cause an excessive amount of DMPs to be concentrated in a small area on the bottom of the well, which can hinder binding with the surface-immobilized cAb. To minimize the assay time, 1 min was selected as the optimal magnetic concentration duration. Sample-DMP and post-magnetic incubation times were then optimized to allow for completion of the assay protocol within 30 min. Experiments to optimize the sample-DMP incubation duration were carried out using 4, 9, and 14 min of sample-DMP incubation with a 5 min post-magnetic concentration incubation time. As expected, longer sample-DMP incubation durations generated larger absorbance values since a longer incubation time allow for more antigen-antibody interactions and more antigen-DMP immunocomplex formation (FIG. 18 Panel C). Using 14 min as the sample-DMP incubation duration, experiments were performed to optimize the post-magnetic concentration incubation duration using 1, 5, and 10 min. The absorbance values generated with 5 and 10 min of post-magnetic concentration incubation were similar for all PfHRP2 concentrations and significantly larger than the absorbance values generated using 1 min of post-magnetic concentration incubation (FIG. 18 Panel D). Therefore, to minimize assay time, 5 min was selected as the optimal post-magnetic concentration incubation duration.

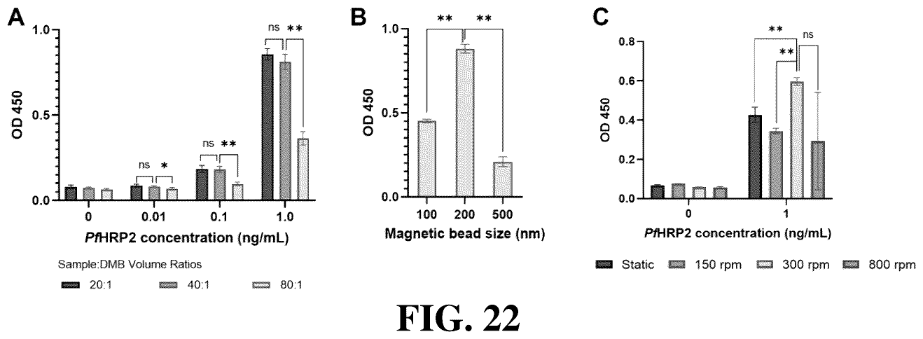

The DMPs and incubation conditions were further optimized to maximize the signal generated by the assay. The amount of DMPs added to the sample was optimized by performing measurements of human serum spiked with PfHRP2 using varying sample-to-DMP solution volume ratios, as shown in FIG. 22 Panel A. The 40:1 and 20:1 sample-to-DMP volume ratios showed no significant difference, but both produced significantly higher absorbance values than those generated using the 80:1 sample-to-DMP volume ratio at all PfHRP2 concentrations, indicating that the 40:1 ratio offers a sufficient amount of DMPs for immunocomplex formation for up to 1 ng mL⁻¹ of target, while minimizing the consumption of magnetic particles and biochemicals. Additionally, the influence of the magnetic bead size on immunomagnetic enrichment performance was investigated by testing PfHRP2-spiked serum samples using DMPs with varying diameters of 100 nm, 200 nm, and 500 nm. It was observed that the 500 nm DMPs concentrated very quickly (within ˜20 sec) at the bottom of the wells; however, they were concentrated within a very small area at the center of the well, limiting their ability to bind to the cAb along the periphery of the well. For this reason, the 500 nm DMPs produced very low absorbance values. The absorbance values generated by the 100 nm DMPs were significantly lower than those generated using the 200 nm DMPs, which is attributed to the reduced magnetic force experienced by the smaller particle, thus requiring a much longer time for adequate magnetic concentration (FIG. 22 Panel B). The optimal conditions for sample-DMP incubation were investigated and found that incubation with agitation at 300 rpm resulted in the largest SBR and smallest variability in the absorbance values compared with faster or slower agitation speeds, or no agitation (FIG. 22 Panel C).

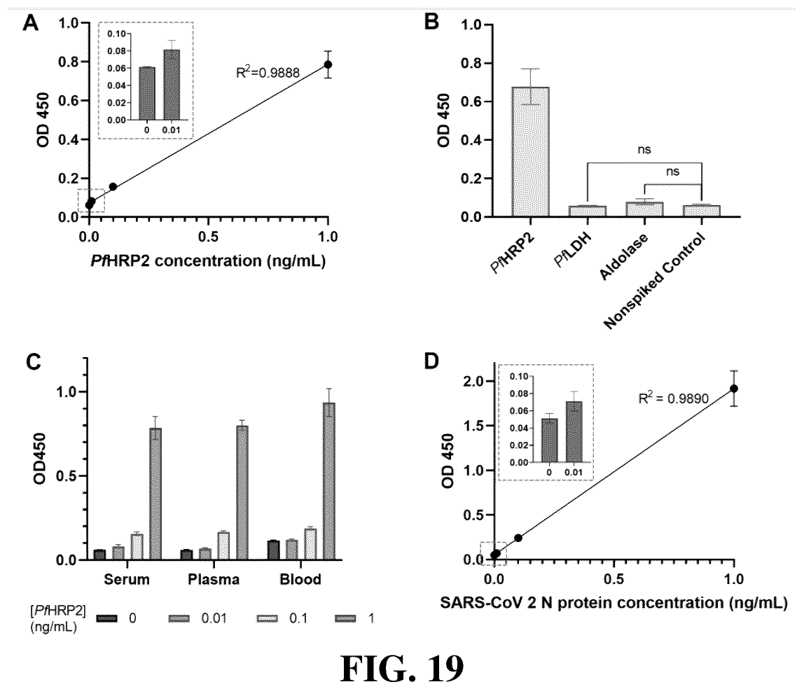

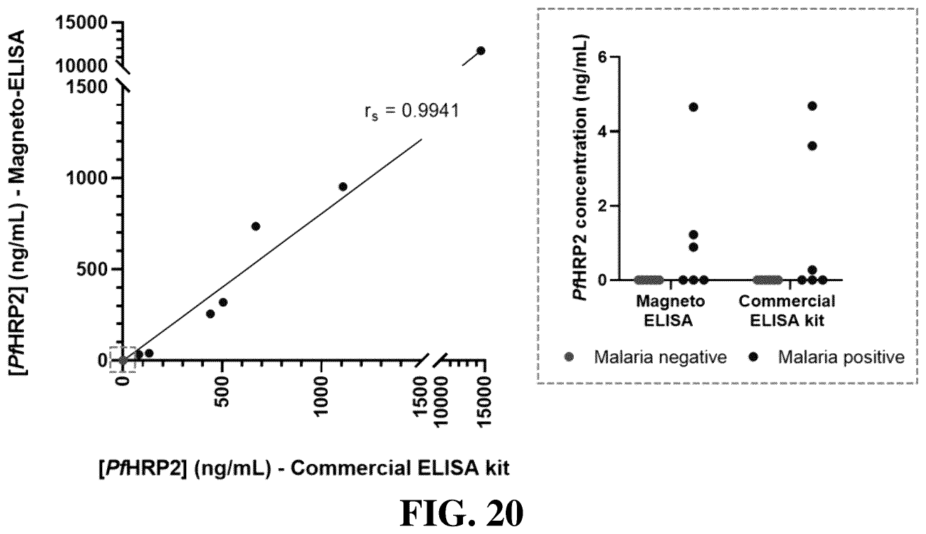

Evaluation of the magneto-ELISA performance: The analytical performance of the magneto-ELISA was first assessed by performing measurements of 10×-diluted human serum spiked with increasing concentration of PfHRP2 from 0 to 1 ng mL⁻¹. The calibration curve, generated from absorbance values at different PfHRP2 concentrations, is shown in FIG. 19 Panel A, which reveals that this assay exhibits a highly linear response (R²=0.9888) from 0 to 1 ng mL⁻¹. The lower LOD of this assay (calculated as 3× standard deviation of the background signal divided by the slope of the linear regression of the calibration curve [62]) is 2 pg mL⁻¹ (33 fM), which is similar to the most sensitive ELISA kits that are commercially available [63, 64]. The specificity of this assay was evaluated by performing measurements of human serum samples spiked with 1 ng mL⁻¹ of PfHRP2, PfLDH or Plasmodium aldolase, and nonspiked serum. As shown in FIG. 19 Panel B, samples containing PfLDH and aldolase resulted in very low absorbance values (˜0.065) and were similar to those generated by nonspiked serum, which was used as the blank control. In contrast, the absorbance values for the sample containing PfHRP2 were 10-fold larger, which indicates that this assay is highly specific to PfHRP2 and will not cross-react with other Plasmodium proteins.

The capability of this assay to detect protein biomarkers in other types of biofluids was investigated by performing measurements of PfHRP2 spiked in 10×-diluted plasma or 10×-diluted whole blood. As shown in FIG. 19 Panel C, the absorbance values produced in plasma and whole blood are similar to those generated in serum with LODs of 10.7 and 12.3 pg mL⁻¹, respectively. The slightly lower analytical sensitivities obtained in plasma and whole blood compared with serum is likely due to the greater background signal produced by nonspecific binding with the additional molecules and proteins found in plasma and blood. These results indicate that this magneto-ELISA can be used for highly sensitive measurements using both purified and whole blood samples.

Lastly, the capability of this assay to detect other protein biomarkers, for instance, the SARS-CoV-2 nucleocapsid (N) protein, was investigated by replacing the anti-PfHRP2 antibodies with anti-SARS-CoV-2 N protein antibodies using the previously optimized parameters. As shown in FIG. 19 Panel D, a highly linear (R²=0.9890) response is also obtained for measurements of the SARS-CoV-2 N protein in human serum with a lower LOD of 8 pg mL⁻¹ (174 fM). Based on these results, it is expected that this magneto-ELISA can be readily adapted for rapid, ultrasensitive measurements of a broad range of clinically relevant protein biomarkers.

Validation of the magneto-ELISA using clinical blood sample: The accuracy of the magneto-ELISA was evaluated by performing measurements of blood samples from individuals with microscopy-confirmed P. falciparum infection and individuals with a negative malaria rapid diagnostic test (RDT) result. PfHRP2 measurements were performed on paired samples using the magneto-ELISA and a commercial Quantimal Ultrasensitive PfHRP2 ELISA kit. Using the calibration curves obtained from measurements of spiked serum samples, PfHRP2 concentrations were determined for the clinical samples using the magneto-ELISA and compared with those determined by the commercial kit. As shown in FIG. 5, the calculated levels of PfHRP2 in the malaria-positive samples range from ˜0.8 ng mL⁻¹ to 11 μg mL⁻¹ and were 0 ng mL⁻¹ for all the malaria RDT-negative samples. The concentrations detected by the magneto-ELISA and commercial kit were highly correlated (Spearman's rank coefficient of 0.9941, p<0.0001), indicating that the magneto-ELISA offers high accuracy. Furthermore, both assays demonstrate the same diagnostic accuracy in identifying PfHRP2-positive and PfHRP2-negative samples and a specificity of 100% for the sample subset used in the experiment. While offering similar diagnostic accuracy as the commercial ELISA kit, this magneto-ELISA is 4× faster (30 min vs. 2 hr incubation time), requires only one washing step, and does not require 37° C. incubation, making it simpler to perform.

A rapid magneto-ELISA for ultrasensitive measurements of protein biomarkers in clinical specimens has been developed as disclosed herein. This was achieved by utilizing DMPs and a simple immunomagnetic enrichment technique, which accelerates the transport of antigen-DMP conjugates to the cAb-immobilized surface, resulting in enhanced signal amplification. The analytical performance of this assay was evaluated by performing measurements of human serum samples spiked with PfHRP2 or SARS-CoV-2 N protein, which exhibited LODs of 2 pg mL⁻¹ and 8 pg mL⁻¹, respectively. In addition to its capability to detect different types of protein biomarkers, measurements of PfHRP2 spiked in human plasma, sera and whole blood demonstrate that this assay is capable of high sensitivity measurements using both purified and whole blood samples. Measurements of PfHRP2 in clinical blood specimens from malaria-positive and malaria-negative individuals reveal that this magneto-ELISA offers similar diagnostic accuracy as a commercial ELISA kit while being 4× faster and simpler to perform. Furthermore, this assay requires no specialized instrumentation and is compatible with standard microplate readers and ELISA protocol, allowing it to integrate readily into current clinical practice for on-site diagnostic testing and blood screening.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
[1] C. W J, S. D S, R. T L, D. K M, C. D E, Y. J J, P. J A, A. G L, N. Engl. J. Med. 1991, 324, 1156-1161.
[2] A. S. Maisel, J. Koon, P. Krishnaswamy, R. Kazenegra, P. Clopton, N. Gardetto, R. Morrisey, A. Garcia, A. Chiu, A. De Maria, Am. Heart J. 2001, 141, 367-374.
[3] B. W E, H. MY, M. A, R. P, Pediatrics 1998, 102, DOI 10.1542/PEDS.102.4.E41.
[4] H. NOEDL, K. YINGYUEN, A. LAOBOONCHAI, M. FUKUDA, J. SIRICHAISINTHOP, R. S. MILLER, Am. J. Trop. Med. Hyg. 2006, 75, 1205-1208.
[5] J. D. Doecke, S. M. Laws, N. G. Faux, W. Wilson, S. C. Burnham, C.-P. Lam, A. Mondal, J. Bedo, A. I. Bush, B. Brown, K. De Ruyck, K. A. Ellis, C. Fowler, V. B. Gupta, R. Head, S. L. Macaulay, K. Pertile, C. C. Rowe, A. Rembach, M. Rodrigues, R. Rumble, C. Szoeke, K. Taddei, T. Taddei, B. Trounson, D. Ames, C. L. Masters, R. N. Martins, for the A. D. N. I. and A. I. B. and L. R. Group, Arch. Neurol. 2012, 69, 1318-1325.
[6] S. S, W. D, B.-B. N, T. S, L. C, PLoS One 2015, 10, DOI 10.1371/JOURNAL.PONE.0143080.
[7] J. Kuhle, H. Kropshofer, D. A. Haering, U. Kundu, R. Meinert, C. Barro, F. Dahlke, D. Tomic, D. Leppert, L. Kappos, Neurology 2019, 92, e1007-e1015.
[8] J. M. Llovet, C. E. A. Peña, C. D. Lathia, M. Shan, G. Meinhardt, J. Bruix, Clin. Cancer Res. 2012, 18, 2290-2300.
[9] S. K. Vashist, J. H. T. Luong, Handb. Immunoass. Technol. Approaches, Performances, Appl. 2018, 1-18.
[10] C. Dincer, R. Bruch, E. Costa-Rama, M. T. Fernández-Abedul, A. Merkoçi, A. Manz, G. A. Urban, F. Güder, Adv. Mater. 2019, 31, 1806739.
[11] S. D. Gan, K. R. Patel, J. Invest. Dermatol. 2013, 133, 1-3.
[12] D. Y. Joh, A. M. Hucknall, Q. Wei, K. A. Mason, M. L. Lund, C. M. Fontes, R. T. Hill, R. Blair, Z. Zimmers, R. K. Achar, D. Tseng, R. Gordan, M. Freemark, A. Ozcan, A. Chilkoti, Proc. Natl. Acad. Sci. 2017, 114, E7054-E7062.
[13] U. Zupančič, P. Jolly, P. Estrela, D. Moschou, D. E. Ingber, Adv. Funct. Mater. 2021, 31, 2010638.
[14] A. Minopoli, B. Della Ventura, B. Lenyk, F. Gentile, J. A. Tanner, A. Offenhäusser, D. Mayer, R. Velotta, Nat. Commun. 2020, 11, 1-10.
[15] A. Hatch, A. E. Kamholz, K. R. Hawkins, M. S. Munson, E. A. Schilling, B. H. Weigl, P. Yager, Nat. Biotechnol. 2001 195 2001, 19, 461-465.
[16] O. Hofmann, G. Voirin, P. Niedermann, A. Manz, Anal. Chem. 2002, 74, 5243-5250.
[17] I. Pereiro, A. F. Khartchenko, R. D. Lovchik, G. V. Kaigala, Angew. Chemie Int. Ed. 2021, DOI 10.1002/ANIE.202107424.
[18] D. Du, J. Wang, D. Lu, A. Dohnalkova, Y. Lin, Anal. Chem. 2011, 83, 6580-6585.
[19] I. F. Cheng, H. L. Yang, C. C. Chung, H. C. Chang, Analyst 2013, 138, 4656-4662.
[20] R. Vaidyanathan, S. Rauf, Y. S. Grewal, L. J. Spadafora, M. J. A. Shiddiky, G. A. Cangelosi, M. Trau, Anal. Chem. 2015, 87, 11673-11681.
[21] H. Cui, C. Cheng, X. Lin, J. Wu, J. Chen, S. Eda, Q. Yuan, Sensors Actuators, B Chem. 2016, 226, 245-253.
[22] Y. Lu, T. Liu, A. C. Lamanda, M. L. Y. Sin, V. Gau, J. C. Liao, P. K. Wong, J. Lab. Autom. 2015, 20, 611-620.
[23] A. Ramos, H. Morgan, N. G. Green, A. Castellanos, J. Phys. D. Appl. Phys. 1998, 31, 2338-2353.
[24] Y. Lu, Q. Ren, T. Liu, S. L. Leung, V. Gau, J. C. Liao, C. L. Chan, P. K. Wong, Int. J. Heat Mass Transf. 2016, 98, 341-349.
[25] M. Sigurdson, D. Wang, C. D. Meinhart, Lab Chip 2005, 5, 1366-1373.
[26] T. Liu, Y. Lu, V. Gau, J. C. Liao, P. K. Wong, Ann. Biomed. Eng. 2014, 42, 2314.
[27] W. C. Lee, H. Lee, J. Lim, Y. J. Park, Appl. Phys. Lett. 2016, 109, 223701.
[28] L. Reverté, B. Prieto-Simón, M. Campàs, Anal. Chim. Acta 2016, 908, 8-21.
[29] S. Liébana, D. Brandão, S. Alegret, M. I. Pividori, Anal. Methods 2014, 6, 8858-8873.
[30] J. Min, M. Nothing, B. Coble, H. Zheng, J. Park, H. Im, G. F. Weber, C. M. Castro, F. K. Swirski, R. Weissleder, H. Lee, ACS Nano 2018, 12, 3378-3384.
[31] B. A. Otieno, C. E. Krause, A. L. Jones, R. B. Kremer, J. F. Rusling, Anal. Chem. 2016, 88, 9269-9275.
[32] A. Valverde, V. Serafín, J. Garoz, A. Montero-Calle, A. González-Cortés, M. Arenas, J. Camps, R. Barderas, P. Yáñez-Sedeño, S. Campuzano, J. M. Pingarrón, Sensors Actuators, B Chem. 2020, 314, 128096.
[33] J. Li, P. B. Lillehoj, ACS Sensors 2021, 6, 1270-1278.
[34] R. W. Snow, BMC Med. 2015 131 2015, 13, 1-3.
[35] I. Pereiro, A. Fomitcheva-Khartchenko, G. V. Kaigala, Anal. Chem. 2020, 92, 10187-10195.
[36] J. Grandke, U. Resch-Genger, W. Bremser, L. A. Garbe, R. J. Schneider, Anal. Methods 2012, 4, 901-905.
[37] C. Selby, Interference in Immunoassay, 1999.
[38] M. L. Chiu, W. Lawi, S. T. Snyder, P. K. Wong, J. C. Liao, V. Gau, J. Assoc. Lab. Autom. 2010, 15, 233-242.
[39] M. Gudmundsson, A. Bjelle, http://dx.doi.org/10.1177/000331979304400507 2016, 44, 384-391.
[40] D. D. Van Slyke, J. Biol. Chem. 1921, 48, 153-176.
[41] A. Castellanos, A. Ramos, A. González, N. G. Green, H. Morgan, J. Phys. D. Appl. Phys. 2003, 36, 2584.
[42] M. L. Y. Sin, T. Liu, J. D. Pyne, V. Gau, J. C. Liao, P. K. Wong, Anal. Chem. 2012, 84, 2702-2707.
[43] M. Kawamura, A. Kusano, A. Furuya, N. Hanai, H. Tanigaki, A. Tomita, A. Horiguchi, K. Nagata, T. Itazawa, Y. Adachi, Y. Okabe, T. Miyawaki, H. Kohno, J. Clin. Lab. Anal. 2012, 26, 174-183.
[44] N. B. Tiscione, K. Wegner, J. Anal. Toxicol. 2017, 41, 313-317.
[45] C. Klumpp-Thomas, H. Kalish, M. Drew, S. Hunsberger, K. Snead, M. P. Fay, J. Mehalko, A. Shunmugavel, V. Wall, P. Frank, J. P. Denson, M. Hong, G. Gulten, S. Messing, J. Hicks, S. Michael, W. Gillette, M. D. Hall, M. J. Memoli, D. Esposito, K. Sadtler, Nat. Commun. 2021 121 2021, 12, 1-13.
[46] T. A. Boyd, P. S. Eastman, D. H. Huynh, F. Qureshi, E. H. Sasso, R. Bolce, J. Temple, J. Hillman, D. L. Boyle, A. Kavanaugh, Correlation of serum protein biomarkers with disease activity in psoriatic arthritis, Expert Rev Clin Immunol, 16 (2020) 335-341.
[47] G. L. Hortin, S. A. Carr, N. L. Anderson, Introduction: Advances in protein analysis for the clinical laboratory, Clin Chem, 56 (2010) 149-151.
[48] J. S. Kang, M. H. Lee, Overview of therapeutic drug monitoring, Korean J Intern Med, 24 (2009) 1-10.
[49] A. D. Powers, S. P. Palecek, Protein analytical assays for diagnosing, monitoring, and choosing treatment for cancer patients, J Healthc Eng, 3 (2012) 503-534.
[50] S. Zhang, A. Garcia-D'Angeli, J. P. Brennan, Q. Huo, Predicting detection limits of enzyme-linked immunosorbent assay (ELISA) and bioanalytical techniques in general, Analyst, 139 (2014) 439-445.
[51] W.H. Organization, The selection and use of essential in vitro diagnostics: report of the third meeting of the WHO Strategic Advisory Group of Experts on In Vitro Diagnostics, 2020 (including the third WHO model list of essential in vitro diagnostics). Geneva: World Health Organization; 2021 (WHO Technical Report Series, No. 1031). Licence: CC BY-NC-SA 3.0 IGO., 2020.
[52] C. K. Dixit, S. K. Vashist, F. T. O'Neill, B. O'Reilly, B. D. MacCraith, R. O'Kennedy, Development of a High Sensitivity Rapid Sandwich ELISA Procedure and Its Comparison with the Conventional Approach, Analytical Chemistry, 82 (2010) 7049-7052.
[53] Y. Gao, Y. Zhou, R. Chandrawati, Metal and Metal Oxide Nanoparticles to Enhance the Performance of Enzyme-Linked Immunosorbent Assay (ELISA), ACS Applied Nano Materials, 3 (2020) 1-21.
[54] A. Ambrosi, F. Airò, A. Merkoçi, Enhanced Gold Nanoparticle Based ELISA for a Breast Cancer Biomarker, Analytical Chemistry, 82 (2010) 1151-1156.
[55] L. F. Huergo, K. A. Selim, M. S. Conzentino, E. C. M. Gerhardt, A. R. S. Santos, B. Wagner, J. T. Alford, N. Deobald, F. O. Pedrosa, E. M. de Souza, M. B. Nogueira, S. M. Raboni, D. Souto, F. G. M. Rego, D. L. Zanette, M. N. Aoki, J. M. Nardin, B. Fornazari, H. M. P. Morales, V. A. Borges, A. Nelde, J. S. Walz, M. Becker, N. Schneiderhan-Marra, U. Rothbauer, R. A. Reis, K. Forchhammer, Magnetic Bead-Based Immunoassay Allows Rapid, Inexpensive, and Quantitative Detection of Human SARS-CoV-2 Antibodies, ACS Sensors, 6 (2021) 703-708.
[56] A. V. Petrakova, A. E. Urusov, A. V. Zherdev, B. B. Dzantiev, Magnetic ELISA of aflatoxin B1—pre-concentration without elution, Analytical Methods, 7 (2015) 10177-10184.
[57] W. Wang, J. Li, C. Dong, Y. Li, Q. Kou, J. Yan, L. Zhang, Ultrasensitive ELISA for the detection of hCG based on assembled gold nanoparticles induced by functional polyamidoamine dendrimers, Analytica Chimica Acta, 1042 (2018) 116-124.
[58] E. de la Serna, K. Arias-Alpízar, L. N. Borgheti-Cardoso, A. Sanchez-Cano, E. Sulleiro, F. Zarzuela, P. Bosch-Nicolau, F. Salvador, I. Molina, M. Ramírez, X. Fernàndez-Busquets, A. Sánchez-Montalvá, E. Baldrich, Detection of Plasmodium falciparum malaria in 1 h using a simplified enzyme-linked immunosorbent assay, Analytica Chimica Acta, 1152 (2021) 338254.
[59] M. de Souza Castilho, T. Laube, H. Yamanaka, S. Alegret, M. I. Pividori, Magneto Immunoassays for Plasmodium falciparum Histidine-Rich Protein 2 Related to Malaria based on Magnetic Nanoparticles, Analytical Chemistry, 83 (2011) 5570-5577.
[60] A. Sánchez-Cano, G. Ruiz-Vega, S. Vicente-Gómez, E. de la Serna, E. Sulleiro, I. Molina, A. Sánchez-Montalvá, E. Baldrich, Development of a Fast Chemiluminescent Magneto-Immunoassay for Sensitive Plasmodium falciparum Detection in Whole Blood, Analytical Chemistry, 93 (2021) 12793-12800.
[61] J. Li, P. B. Lillehoj, Microfluidic Magneto Immunosensor for Rapid, High Sensitivity Measurements of SARS-CoV-2 Nucleocapsid Protein in Serum, ACS Sens, 6 (2021) 1270-1278.
[62] D. C. Harris, Quantitative Chemical Analysis, 7 ed., W. H. Freeman and Co., New York, NY, 2007.
[63] S. Tang, I. Hewlett, Nanoparticle-based immunoassays for sensitive and early detection of HIV-1 capsid (p24) antigen, The Journal of Infectious Diseases, 201 (2010) S59-S64.
[64] M. V. Tsapenko, R. E. Nwoko, T. M. Borland, N. V. Voskoboev, A. Pflueger, A. D. Rule, J. C. Lieske, Measurement of urinary TGF-β1 in patients with diabetes mellitus and normal controls, Clin Biochem, 46 (2013) 1430-1435.
[65] Fabiani, L., “Magnetic Beads Combined with Carbon Black-Based Screen-Printed Electrodes for COVID-19: A Reliable and Miniaturized Electrochemical Immunosensor for SARS-CoV-2 Detection in Saliva,” Biosens. Bioelectron. 2021, 171, 112686.
[66] Tan, X., “Rapid and Quantitative Detection of SARS-CoV-2 Specific IgG for Convalescent Serum Evaluation,” Biosens. Bioelectron. 2020, 169, 112572.
[67] Torrente-Rodríguez, R. M., “SARS-CoV-2 RapidPlex: A Graphene-Based Multiplexed Telemedicine Platform for Rapid and Low-Cost COVID-19 Diagnosis and Monitoring,” Matter 2020, 3, 1981-1998.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto and their equivalents. The entirety of each of the aforementioned documents is incorporated by reference herein.

1. A microfluidic method for detecting a target protein in a sample comprising:
(a) contacting the sample with immunosensors comprising dually-labeled magnetic beads (DMBs) conjugated to a capture antibody specific for the target protein and an enzyme reporter;
(b) loading the sample and DMBs into a microfluidic chip;
(c) applying AC electrothermal flow (ACEF) to the sample to mix the sample;
(d) performing immunomagnetic enrichment to generate an electrochemical signal; and
(e) detecting the target protein by measuring levels of the reporter.

2. The method of claim 1, wherein the capture antibody is a human monoclonal capture antibody.

3. The method of claim 1, wherein the sample to DMBs ratio is about 10:1 to about 20:1.

4. The method of claim 1, wherein contacting is for about 40 minutes to about 60 minutes.

5-7. (canceled)

8. The method of claim 1, wherein the sample and DMBs are loaded onto the microfluidic chip using a capillary tube and plunger or a syringe pump.

9. (canceled)

10. The method of claim 1, wherein the reporter generates an electrochemical signal or an optical signal.

11. (canceled)

12. The method of claim 1, wherein the reporter is a chemiluminescent reporter.

13. (canceled)

14. The method of claim 13, wherein measuring levels of the reporter comprises using an HRP-conjugated detection antibody and detecting colorimetric signal.

15-18. (canceled)

19. The method of claim 16, wherein measuring levels of the reporter comprise detecting amperometric current.

20. (canceled)

21. The method of claim 1, wherein the ACEF is applied at about 200 kHz and 25 Vpp.

22. The method of claim 1, wherein the ACEF is applied for about 5 minutes.

23-27. (canceled)

28. The method of claim 1, wherein the method does not comprise centrifugation of the sample.

29-32. (canceled)

33. The method of claim 1, wherein the method is performed in less than 30 minutes.

34. (canceled)

35. The method of claim 1, wherein the sample volume is less than 50 uL.

36. (canceled)

37. A device for quantitative measurements of a target protein in a sample, wherein the device is a handheld diagnostic comprising:
a microfluidic chip with an immunosensor; and
a magnet proximal to the immunosensor.

38. The device of claim 37, wherein the microfluidic chip further comprises: an inlet and a sample loading mechanism; an outlet; and a waste reservoir.

39-40. (canceled)

41. The device of claim 37, wherein the immunosensor comprises a working electrode, a counter electrode and a reference electrode.

42. The device of claim 41, wherein the device is configured to provide mixing to a sample via alternating current electrothermal flow (ACEF).

43. The device of claim 42, further comprising a detector configured to detect a signal from the immunosensor.

44-48. (canceled)

49. A microfluidic electrochemical magneto-immunosensor for rapid and high sensitivity measurements of protein biomarkers in biofluid samples, wherein the assay is based on a sensing scheme utilizing dually labeled magnetic nanobeads for immunomagnetic enrichment and signal amplification.

50. (canceled)
